(19) United States
(12) Patent Application Publication
(10) Pub. No.: US 2024/0366766 A1
(43) Pub. Date: Nov. 7, 2024

(54) POLYMER COMPOUND FOR SURFACE MODIFICATION TO ENHANCE ANTICANCER IMMUNE FUNCTION OF NATURAL KILLER CELLS

(71) Applicant: DONGGUK UNIVERSITY INDUSTRY-ACADEMIC COOPERATION FOUNDATION, Seoul (KR)

(72) Inventors: Kyobum KIM, Seoul (KR); Sungjun KIM, Seoul (KR); Gajendiran MANI, Seoul (KR)

(21) Appl. No.: 18/641,336

(22) Filed: Apr. 20, 2024

Related U.S. Application Data
(63) Continuation of application No. PCT/KR2022/016204, filed on Oct. 21, 2022.

(30) Foreign Application Priority Data
Oct. 22, 2021 (KR) 10-2021-0141920

(51) Int. Cl. A61K 47/32; A61K 35/17; A61K 47/10; A61K 47/18; A61P 35/00; C08L 81/10

(52) U.S. Cl. CPC A61K 47/32; A61K 35/17; A61K 47/10; A61K 47/183; A61P 35/00; C08L 81/10

(57) ABSTRACT

Provided relates to a polymer compound and a method for preparing same, the polymer compound comprising: a hydrophobic moiety that binds to natural killer cells; a cancer cell recognition moiety; and a linker, wherein the hydrophobic moiety is bound to one end of the linker, and the cancer cell recognition moiety is bound to the other end of the linker, thus recognizing natural killer cells and cancer cells.

### TECHNICAL FIELD

The present invention relates to a polymer compound synthesized and manufactured to attach to the surface of natural killer cells, enhancing their anti-cancer function. By injecting the synthesized polymer compound into the body, it enhances the recognition and elimination of cancer cells, thereby significantly improving the effectiveness of cancer prevention or treatment.

### BACKGROUND ART

The administration of cytokine protein preparations such as interleukin or interferon to enhance existing immune responses and induce the activation of effector cells may lead to the potential occurrence of systemic immunotoxicity side effects.

Additionally, the presence of immune-suppressive microenvironment factors, including T-cell activity inhibitors secreted by tumor tissues, may hinder the application of conventional methods for enhancing immune responses and inducing effector cell activation in solid tumors where T cells are difficult to reach.

As a solution to enhance the activity of immune response and effector cells, cell coating technology may be introduced. However, it comes with certain limitations.

The cell coating technology mentioned above can utilize the layer-by-layer assembly method. This layer-by-layer assembly method is a widely applicable, simple, and versatile deposition process used to address various issues such as biomolecule deposition, concentration, biological activity, coating thickness, and release rate.

However, to utilize the mentioned layer-by-layer assembly method, surface modification of the cells is necessary, which poses challenges in the process due to the alternating reaction of cationic or anionic organic substances for stacking. Moreover, there is a risk of functional impairment of surface membrane proteins involved in signal transduction and cancer cell recognition due to the complete coverage of the cell surface with coating materials. Additionally, in cases where cell surface coating is used for a single purpose such as preventing cell aggregation, there is often a lack of strategies for multifunctionality that can lead to an effective immune anti-cancer therapy.

The inventors have synthesized polymer compounds that coat the surface of natural killer cells, thus completing the present invention. These compounds not only prevent the functional impairment of surface membrane proteins involved in signal transduction and cancer cell recognition but also exhibit anti-cancer effects.

### DISCLOSURE

### Technical Problems

The technical challenge that the present invention aims to address is to enhance the anti-cancer functionality of natural killer cells through a surface attachment single process. This process allows for the partial attachment of natural killer cells to localized areas on the surface, thereby preventing the functional decrease of cell signaling membrane proteins. Furthermore, the invention provides multifunctional polymers capable of maintaining the innate functionality of natural killer cells and reducing side effects through a responsive unmasking process based on the reductive conditions of cancer cells or tumor microenvironments.

### Technical Solution

As an embodiment of the present invention aimed at addressing the aforementioned challenge, a polymer compound recognizing natural killer cells and cancer cells may include a hydrophobic moiety capable of binding to natural killer cells, a cancer cell recognition moiety, and a linker having the structure represented by Chemical Formula 1. At one end of the linker, the hydrophobic moiety may be bound, while at the other end of the linker, the cancer cell recognition moiety may be bound.

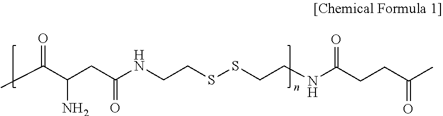

Here, the above ‘n’ is an integer ranging from 30 to 50.

As an embodiment of the present invention, a polymer compound capable of recognizing natural killer cells and cancer cells may include a hydrophobic moiety and a cancer cell recognition moiety. The hydrophobic moiety can bind to natural killer cells, while the cancer cell recognition moiety can recognize cancer cells and promote their elimination.

As an embodiment of the present invention, a polymer compound capable of recognizing natural killer cells and cancer cells may include a hydrophobic moiety that binds to the natural killer cells. The hydrophobic moiety binding to the natural killer cells may be an lipid moiety with a alkyl chain of 12 to 24 carbon atoms; a sterol lipid with 10 to 30 carbon atoms; 1,2-bis(diphenylphosphino)ethane (DPPE); or 1,2 bis (dimethylphosphino)ethane (DMPE).

As an embodiment of the present invention, the polymer compound capable of recognizing natural killer cells and cancer cells may include a cancer cell recognition moiety.

The cancer cell recognition moiety may be selected from the group consisting of folic acid, biotin, phenylboronic acid, and phenylboronic acid.

As an embodiment of the present invention, the polymer compound capable of recognizing natural killer cells and cancer cells may include a linker comprising the structure of Formula 1, to which one or more compounds for preventing cellular internalization, cationic amino acids, and fluorescent dye compounds may be attached.

As an embodiment of the present invention, in the polymer compound capable of recognizing natural killer cells and cancer cells, the compound for preventing cellular internalization, which is bound to the linker comprising the structure of Formula 1, may include one or more compounds selected from the group consisting of polyethylene glycol (PEG), polyethylene oxide (PEO), polyvinyl alcohol (PVA), and their copolymers.

As an embodiment of the present invention, in the polymer compound capable of recognizing natural killer cells and cancer cells, the cationic amino acid bound to the linker comprising the structure of Formula I may include one or more compounds selected from the group consisting of arginine, lysine, and histidine.

As an embodiment of the present invention, a method for preparing a polymer compound capable of recognizing natural killer cells and cancer cells may include the following steps: (a) providing a compound represented by Formula 3 by coupling a compound represented by Formula 2 to one end of a compound represented by Formula 1;
(b) Optionally, including a step of coupling a cancer cell recognition moiety to the other end of the compound represented by Formula 3.

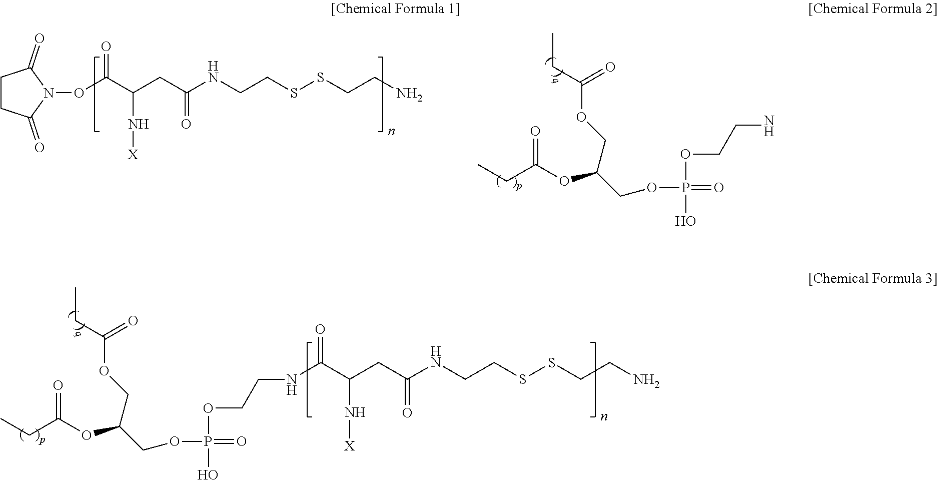

Here, the variable n is an integer ranging from 30 to 50, and X is selected from acetyl, trifluoroacetyl, t-butoxycarbonyl (BOC), benzyl oxycarbonyl (CBZ), and 9-fluorenylmethyloxycarbonyl (Fmoc), and p and q are integers ranging from 12 to 20.

As an embodiment of the present invention, the method for preparing a polymer compound that recognizes natural killer cells and cancer cells may further include a step of substituting X of the compound represented by the chemical formula 1 with a compound for preventing cellular internalization, an amino acid, or a fluorescent dye compound.

As an embodiment of the present invention, in the method for preparing a polymer compound that recognizes natural killer cells and cancer cells, the cancer cell recognition moiety bound to the other end of the compound represented by the chemical formula 3 may be selected from the group consisting of folic acid, biotin, phenylboronic acid, and phenylboronic acid.

As an embodiment of the present invention, in the method for preparing a polymer compound that recognizes natural killer cells and cancer cells, the polymer compound represented by chemical formula 1 may be substituted with a cell internalization prevention compound, which can be polyethylene glycol (PEG), polyethylene oxide (PEO), polyvinyl alcohol (PVA), or a copolymer thereof.

As an embodiment of the present invention, in the method for preparing a polymer compound that recognizes natural killer cells and cancer cells, the cationic amino acid that can be bound to X in the compound represented by chemical formula 1 may be selected from a group consisting of arginine, lysine, and histidine, and may include one or more thereof.

As an embodiment of the present invention, a pharmaceutical composition for cancer prevention or treatment of the present invention may comprise a polymer compound that recognizes natural killer cells and cancer cells as an active ingredient, comprising a hydrophobic moiety binding to natural killer cells, a cancer cell recognition moiety, and a linker including the structure of chemical formula 1, where the hydrophobic moiety is bound to one end of chemical formula 1, and the cancer cell recognition moiety is bound to the other end of chemical formula 1.

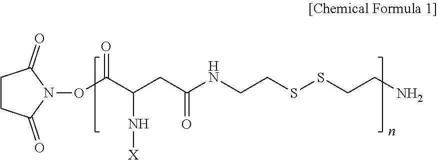

Here, the variable n is an integer ranging from 30 to 50.

As an embodiment of the present invention, diseases in which anticancer effects are achieved by pharmaceutical compositions for cancer prevention or treatment comprising the polymer compound recognizing natural killer cells and cancer cells as active ingredients may include, but are not limited to, prostate cancer, thyroid cancer, stomach cancer, colorectal cancer, lung cancer, breast cancer, liver cancer, pancreatic cancer, testicular cancer, oral cancer, basal cell carcinoma, brain tumors, gallbladder cancer, bile duct cancer, laryngeal cancer, retinoblastoma, sarcoma, bladder cancer, peritoneal cancer, adrenal cancer, non-small cell lung cancer, esophageal cancer, small cell lung cancer, colon cancer, meningioma, esophageal cancer, renal pelvis and ureter cancer, kidney cancer, malignant bone tumors, malignant connective tissue tumors, malignant lymphomas, malignant melanoma, eye tumors, urethral cancer, gastric cancer, lipoma, laryngeal cancer, cervical cancer, endometrial cancer, uterine fibroids, metastatic brain tumors, rectal cancer, vaginal cancer, spinal cord tumors, salivary gland cancer, tonsil cancer, squamous cell carcinoma, blood cancer, and anal cancer.

The term “hematologic cancer” refers to cancers that affect the hematopoietic system, which produces essential blood components in our body, and also includes cancers that affect the immune function, protecting our body from infections and other harmful factors. Specifically, it may encompass leukemia, lymphoma, multiple myeloma, and other cancers originating from lymph nodes and lymphatic organs.

As an embodiment of the present invention, pharmaceutical compositions for cancer prevention or treatment comprising the polymer compound recognizing natural killer cells and cancer cells as active ingredients may further include a pharmaceutically acceptable carrier, excipient, or diluent.

An embodiment of the present invention, a method for treating cancer, may include administering a pharmaceutical composition for cancer prevention or treatment comprising the polymer compound recognizing natural killer cells and cancer cells to a subject.

### Advantageous Effects

The present invention relates to a polymer compound comprising hydrophobic moieties for natural killer cell surface binding and cancer cell recognition moieties, incorporating various functional groups. The surface-modified natural killer cells with the polymer compound described above can significantly increase the cancer cell recognition ability, leading to a notable increase in cancer cell destruction through the apoptotic effect induced by the release of lytic granules and cytokines.

Furthermore, the apoptotic effect on cancer cells can be significantly enhanced overall as a result of the increased cancer cell destruction by the surface-modified natural killer cells. This is facilitated by the continuous binding of the cancer cell recognition moiety to the surface of cancer cells, even after the cancer cells have been destroyed by the released glutathione from the apoptotic cancer cells.

### BRIEF DESCRIPTION OF DRAWINGS

FIG. 1 is a diagram depicts the method for enhancing the immune anticancer function of natural killer cells through the attachment of polymeric compounds for surface modification of natural killer cells according to an embodiment of the present invention.

FIG. 2 is a view of natural killer cells coated with a solution containing the polymer compound, labeled with fluorescent markers, and observed under a fluorescence microscope.

FIG. 3 is a graph representing the fluorescence intensity of the coated material relative to natural killer cells.

FIG. 4 is a graph obtained through flow cytometry analysis, demonstrating the attachment duration of biomolecules coated on the cell membrane surface of natural killer cells for up to 48 hours. The peak range furthest to the right represents the condition where no time has elapsed after attaching the biomolecules to the natural killer cell membrane 0 hr. The next peak range to the left represents 6 hours after attachment, followed by the peak range indicating 12 hours, then 24 hours, and finally 48 hours after attachment, along with the peak range for natural killer cells without coating. Here, the peak range refers to the range encompassing the left and right portions of the graph where peak values are observed.

FIG. 5 is a left graph showing the viability of natural killer cells as a function of coating material concentration, while the graph on the right demonstrates the viability of natural killer cells over time.

FIG. 6 is a graph indicating that after the attachment of biomolecules to the surface, the receptor molecules responsible for signal transduction on the membrane surface of natural killer cells maintain their intrinsic characteristics and are shown to interact appropriately with corresponding ligands.

FIG. 7 is a diagram illustrating the apoptotic effect on cancer cells as a result of the detachment of ligands enhancing cancer cell recognition from the surface of the polymer under the microenvironment conditions of cancer cells/tissues.

FIG. 8 is the chemical structures of intermediates Fmoc-protected aspartate (Fmoc-Asp), cystamine dihydrochloride, and Fmoc-PEDS confirmed by NMR spectra.

FIG. 9 illustrates chemical structures, in which the chemical structure of Fmoc-protected aspartate (Fmoc-Asp) is shown by (A), cystamine dihydrochloride is shown by (B), and the synthesized intermediate Fmoc-PEDS, confirmed by FTIR spectra, is shown by (C).

FIG. 10 illustrates chemical structures, in which the chemical structure of the Fmoc-PEDS intermediate is shown by (A), DSPE lipid is depicted by (B), and the chemical structure of the DSPE-Fmoc-PEDS conjugate, confirmed by NMR spectra, is shown by (C).

FIG. 11 illustrates chemical structures, in which the chemical structure of the Fmoc-PEDS intermediate is shown by (A), DSPE lipid is depicted by (B), and the chemical structure of the DSPE-Fmoc-PEDS conjugate, confirmed by FTIR spectra, is shown by (C).

FIG. 12 illustrates chemical structures, in which the chemical structure of succinoyl-DSPE-Fmoc-PEDS is shown by (A), folic acid (FA) is shown by (B), and the chemical structure of DSPE-PEDS-FA, confirmed by NMR spectra, is shown by (C).

FIG. 13 illustrates chemical structures, in which the chemical structure of succinoyl-DSPE-Fmoc-PEDS is shown by (A), folic acid (FA) is represented by (B), and the chemical structure of DSPE-PEDS-FA, confirmed by FT-IR spectra, is shown by (C).

FIG. 14 illustrates chemical structures, in which the chemical structure of PEG₁ₖ-COOH is shown by (A), Fmoc-arginine is shown by (B), the 5CFL dye is shown by (C), and the synthesized multifunctional lipid-PEDS_{5CFL-Arg-PEG1k}-FA, confirmed by NMR spectra, is shown by (D).

FIG. 15 illustrates chemical structures, in which the chemical structure of DSPE-PEDS-FA is shown by (A), PEG1k-COOH is represented by (B), Fmoc-arginine is shown by (C), the 5CFL dye is illustrated by (D), and the synthesized multifunctional lipid-PEDS5CFL-Arg-PEG1k-FA, confirmed by FT-IR spectra, is displayed by (E).

FIG. 16 illustrates chemical structures, in which the chemical structure of DSPE-PEDS-FA is shown by (a), PEG1-COOH is represented by (b), Fmoc-arginine is shown by (c), the 5CFL dye is illustrated by (d), and the synthesized multifunctional lipid-PEDS5CFL-Arg-PEG1k-FA, confirmed by FT-IR spectra, is displayed by (e).

FIG. 17 illustrates chemical structures, in which the chemical structure of DSPE-PEDS-Biotin is shown by (a), 5-CFL is illustrated by (b), Fmoc-Arg is shown by (c), PEG1k is depicted by (d), and the chemical structure of DSPE-PEDS_{5CFL/Arg/PEG1k}-Biotin, confirmed by FT-IR spectra, is displayed by (e).

FIG. 18, is the graph representing the remaining effector-to-target (E: T) cell ratio, where Effector cells (either NK-92mi cells or coated NK-92mi cells) are compared to Target cells at a ratio of 10:1.

FIG. 19 is the view of the quantification results of DNA content in the aforementioned target cells.

FIG. 20 is the graph representing the specific cell lysis results through cultured NK-92mi cells according to Example 1 of the invention. The x-axis indicates the ratio of effector cells to target cells, while the y-axis represents the percentage of cell lysis and apoptosis.

FIG. 21 is the graph representing the specific cell lysis results through cultured NK-92mi cells according to Example 2 of the invention. The x-axis indicates the ratio of effector cells to target cells, while the y-axis represents the percentage of cell lysis and apoptosis.

FIG. 22 is the graph representing the concentration of glutathione released from each target cell (MCF-7 breast cancer cells, MDA-MB-231 breast cancer cells, MIA PaCa-2 pancreatic cancer cells, or human fibroblast cells).

FIG. 23 is a schematic diagram depicting the experimental process of creating structures mimicking coated natural killer cells anchored through hydrophobic interactions, and using them to confirm the apoptotic effect on cancer cells via folate-mediated pathways.

FIG. 24 is a schematic diagram illustrating the experimental process for confirming the apoptotic effect of coated natural killer cells on cancer cells in vivo.

FIG. 25 is a diagram in which (A) representing hematoxylin and eosin (H&E) stained images of tumor tissues harvested from MDA MB-231 xenograft mice. Here, ‘T’ represents the tumor tissue area, and ‘N’ indicates the necrotic area. White arrows denote eosinophilic cell swelling, white triangles indicate condensed nuclear morphology, and black arrows represent cells with dissolved nuclei. (B) quantifies the necrotic area from the H&E-stained images of the entire tumor mass, where the ratio of necrotic area was significantly higher in the coated NK cell group, reaching up to 36% compared to other groups. (C) depicts the expression level of cleaved caspase-3 as a marker of cell apoptosis, where the aggregated forms within rectangles indicate cells expressing cleaved caspase-3. (D) quantifies cleaved caspase-3 positive cells in tumor tissue, showing a significantly higher ratio, up to 26%, in the coated NK cell group compared to all other groups. (E) presents a picture confirming proliferating tumor cells using the proliferation marker Ki67, with aggregated forms within rectangles indicating areas of tumor cell proliferation. (F) quantifies the proliferating tumor cell portion using the proliferation marker Ki67, showing a ratio approximately one-fifth that of the control group in the coated NK cell group. (G) displays images of CD56 (human-specific NK cell marker) positive cells, with aggregated forms within rectangles representing CD56-positive cells. (H) quantifies the distribution of NK cells or coated NK cells in major organs and tumors based on the ratio of CD56-positive cells, indicating a higher distribution within tumors compared to NK cell groups, especially in the heart, kidney, liver, lungs, and spleen. The proportion of coated NK cells distributed in tumors was higher than that of NK cell groups.

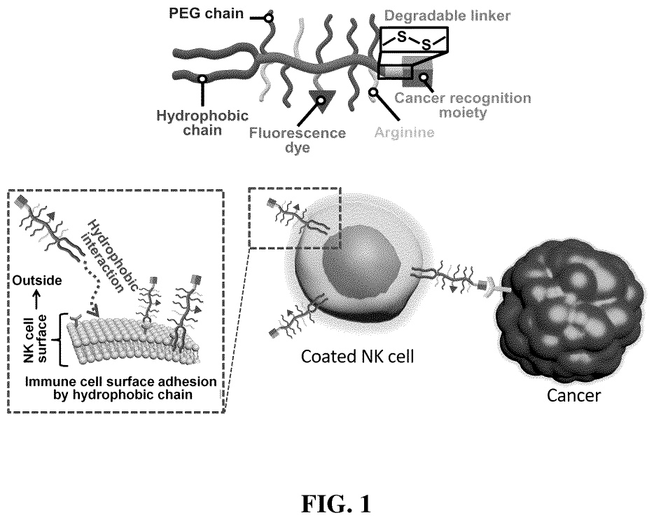

### BEST MODE FOR INVENTION

The following describes the present invention in detail through embodiments and experimental examples. However, it should be noted that the embodiments and experimental examples provided below are merely illustrative of the present invention, and the scope of the invention is not limited by the embodiments and experimental examples presented herein.

### Example 1: Preparation of Polymer Compounds for Surface Modification to Enhance Immune Anticancer Activity of Natural Killer Cells

The components used to manufacture the polymer compounds of the present invention are listed in Table 1.

TABLE 1

| Component | Manufacturing company |
|---|---|
| N-(3-Dimethylaminopropyl)- | Sigma-Aldrich, 03449 |
| N-ethylcarbodiimide | |
| hydrochloride, EDC) | |
| N-hydroxysuccinimide, NHS | Sigma-Aldrich, 130672 |
| cystamine dihydrochloride, Cys | Sigma-Aldrich, 30050 |
| 4-Dimethylaminopyridine, DMAP | Sigma-Aldrich, 39405 |
| N,N-Dimethylformamide, DMF | Sigma-Aldrich, 227056 |
| folic acid, FA | Sigma-Aldrich, F7876 |
| 5-carboxyfluorescein, 5CFL | Sigma-Aldrich, 86826 |
| triethylamine | Sigma-Aldrich, 471283 |
| 1,2-Distearoyl-sn-glycero- | TCI, 850745P |
| 3-phosphoethanolamine DSPE | |
| PEG1k-NHS | Biopharma PEG Scientific Inc., |
| | HE023017 |
| Fmoc-aspartic acid(Fmoc-Asp) | BOC Science, 119062-05-04 |
| Fmoc-arginine(Fmoc-Arg) | BOC Science, 91000-69-0 |
| succinic anhydride | Daejung, 7673-4405 |
| diethyl ether | Duksan, D558 |
| piperidine | Sigma-Aldrich, 104094 |
| biotin | Sigma-Aldrich, B4501 |
| dimethyl sulfoxide | Sigma-Aldrich, 276855 |

### 1-1. Synthesis of Fmoc-PEDS Intermediate

1 mmol of Fmoc-ASP, 2 mmol of EDC, and 3 mmol of NHS were mixed in 5 mL of anhydrous dimethylformamide (DMF), and the reaction mixture was stirred at 25° C. for 3 hours to prepare the reaction mixture.

The solution of cystamine dihydrochloride (concentration of 1 mmol) dissolved in 5 mL of anhydrous dimethylformamide (DMF) and 5 mL of 4-dimethylaminopyridine were added to the above reaction mixture.

The reaction mixture, with cystamine dihydrochloride and 4-dimethylaminopyridine added, was allowed to stand for 15 hours.

1 mmol of EDC, 1 mmol of NHS, and 2 mg of 4-dimethylaminopyridine were dissolved in 1 mL of anhydrous dimethylformamide. This solution was then added to the previously incubated reaction mixture, and the resulting mixture was stirred at 60° C. and 600 rpm using a stirring apparatus (Corning, pC-620D) for 60 hours.

At the point of completion of the reaction, which occurred after 60 hours, the obtained product was precipitated by adding it dropwise into 100 mL of diethyl ether at −20° C. using centrifugation at 12,000 rpm for 10 minutes (LABOGENE, 1580R). This process yielded 334 mg of Fmoc-PEDS (Fmoc-poly (ethylene aspartamido disulfide) intermediate).

The molecular weight of the synthesized 334 mg of Fmoc-PEDS intermediate was measured using a gel permeation chromatography (GPC) system equipped with a GPC column (Styragel) using poly (methyl methacrylate) as the standard.

The chemical structure confirmation of the Fmoc-PEDS intermediate was also confirmed by NMR (500 MHZ FT-NMR spectrometer, Bruker, Germany) and FTIR (Perkin Elmer FTIR Spectrum Two, PerkinElmer, USA) spectroscopic analyses.

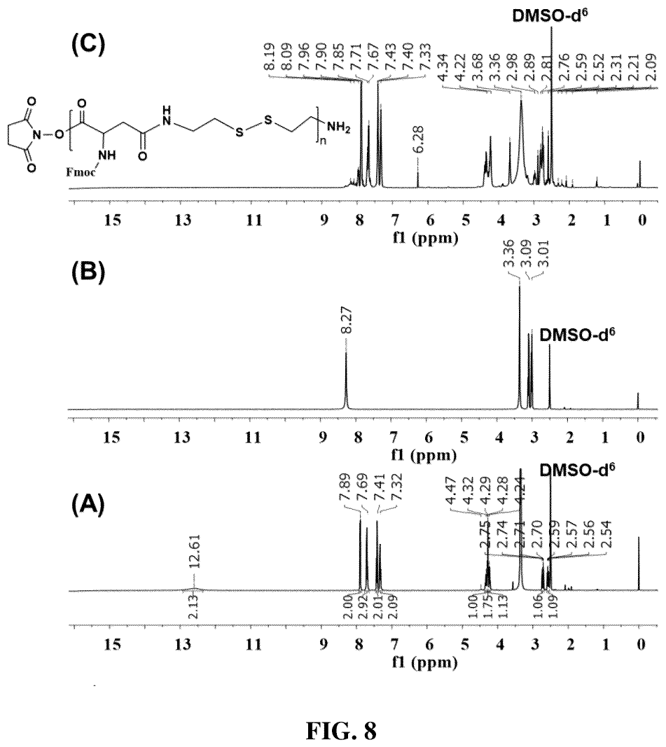

FIG. 8 depicts the chemical structures of Fmoc-protected aspartate (Fmoc-Asp), cystamine dihydrochloride, and the Fmoc-PEDS intermediate as confirmed by NMR spectra.

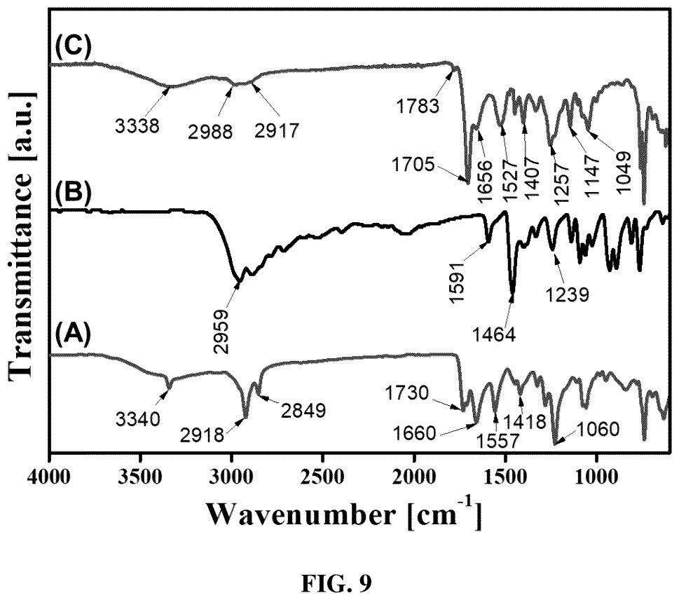

In FIG. 9, the chemical structure of Fmoc-protected aspartate (Fmoc-Asp) is represented in (A), cystamine dihydrochloride is depicted in (B), and the chemical structure of the synthesized Fmoc-PEDS intermediate is shown in (C), as confirmed by FTIR spectra.

The degree of polymerization (DP) was calculated using the following equation.

$$DP = \frac{Molecular \cdot wieght \cdot of \cdot polymer}{Molecular \cdot weight \cdot of \cdot monomer}$$

### 1-2. Formation of DSPE-Fmoc-PEDS Conjugate

1 mmol of EDC, 1.5 mmol of NHS, and 100 mg of the Fmoc-PEDS intermediate synthesized in example 1-1 (containing 4.90 μmol of terminal carboxyl groups) were dissolved in 5 mL of anhydrous dimethylformamide (DMF). The mixture was stirred at 25° C. with a stirring apparatus (Corning, pC-620D) at a speed of 600 rpm for 3 hours to prepare the mixture.

Subsequently, a solution of 20 μmol of DSPE dissolved in 5 mL of 4-dimethylaminopyridine was added to the completed stirred mixture, and the resulting mixture was maintained at 25° C. under nitrogen (N2) conditions for 48 hours to form the reaction mixture.

The reaction mixture was precipitated in 100 mL of diethyl ether at −20° C. and then vacuum-dried at −80° C. to synthesize 100 mg of DSPE-Fmoc-PEDS conjugate (including 4.90 μmol of terminal amine groups).

The confirmation of the chemical structure of the DSPE-Fmoc-PEDS conjugate was also conducted using NMR (500 MHZ FT-NMR spectrometer, Bruker, Germany) and FTIR (Perkin Elmer FTIR Spectrum Two, PerkinElmer, USA) spectroscopic analysis.

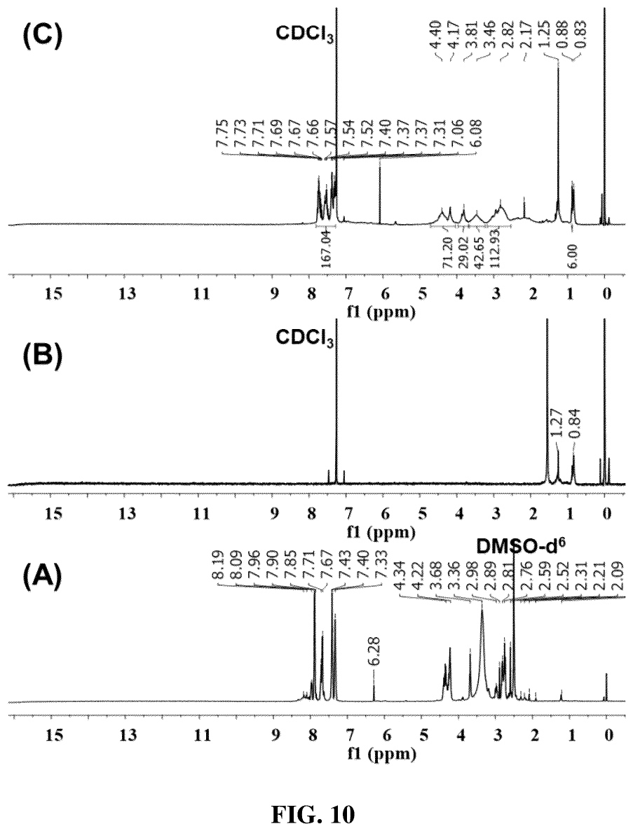

In FIG. 10, the chemical structure of Fmoc-PEDS intermediate is illustrated in (A), the chemical structure of DSPE lipid is illustrated in (B), and the chemical structure of DSPE-Fmoc-PEDS conjugate is illustrated in (C), as confirmed by NMR spectra.

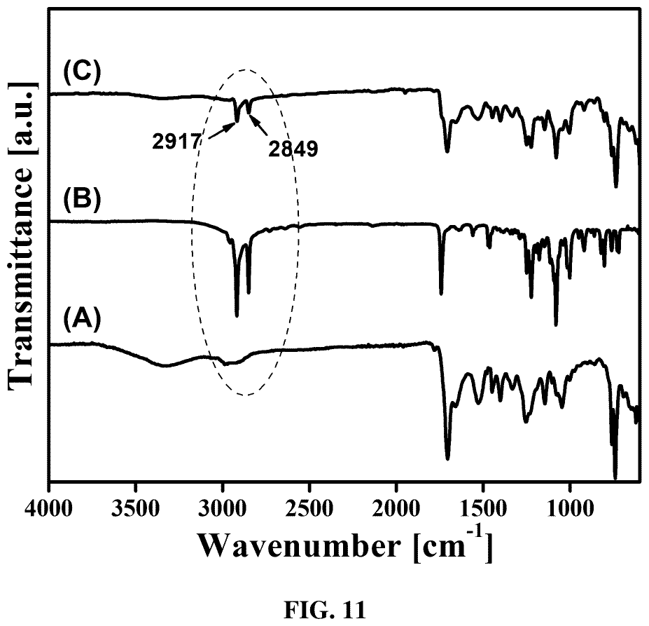

In FIG. 11, the chemical structure of Fmoc-PEDS intermediate is illustrated in (A), the chemical structure of DSPE lipid is illustrated in (B), and the chemical structure of DSPE-Fmoc-PEDS conjugate is illustrated in (C), as confirmed by FTIR spectra.

### 1-3. Synthesis of DSPE-PEDS-FA Conjugate

The 100 mg of DSPE-Fmoc-PEDS synthesized in example 1-2 was dissolved in 5 mL of anhydrous dimethylformamide (DMF), along with 4.9 mg of succinic anhydride (concentration of succinic anhydride: 49 μmol), to prepare the reaction mixture.

The reaction mixture was stirred using a stirring apparatus (Corning, pC-620D) at 25° C. for 24 hours with a stirring speed of 600 rpm to prepare the reaction mixture.

The stirred reaction mixture was precipitated in 100 mL of diethyl ether at −20° C., followed by the addition of 10 mL of distilled water (DW). The mixture was then centrifuged at 10,000 rpm for 10 minutes (LABOGENE, 1580R) to remove the supernatant containing unreacted succinic anhydride (C4H4O3), resulting in the preparation of the reaction mixture.

The reaction mixture, from which succinic anhydride (C4H4O3) had been removed, was subjected to dialysis (fractional molecular weight cutoff 2 kD), followed by freeze-drying at 0° C., resulting in the preparation of 100 mg of succinoyl-DSPE-Fmoc-PEDS conjugate (with an equivalent terminal carboxyl group concentration of 4.90 μmol).

To deprotect the Fmoc moiety and facilitate the conjugation of folic acid (FA), a solution was prepared by dissolving 100 mg of the succinoyl-DSPE-Fmoc-PEDS conjugate (with an equivalent terminal carboxyl group concentration of 4.90 μmol) in 5 mL of anhydrous dimethylformamide (DMF), adjusting the concentration of EDC to 1 mmol and NHS to 1.5 mmol. The resulting solution was then subjected to stirring at 25° C. for 1 hour at a speed of 600 rpm using a stirring device (Corning, pC-620D) to produce the reaction mixture.

5.90 μmol of folic acid (FA) was dissolved in a solvent mixture consisting of 5 mL of 4-dimethylaminopyridine and 5 mL of dimethyl sulfoxide (DMSO). This solution was then mixed with the stirred reaction mixture described above and stirred at 25° C. under a nitrogen (N2) atmosphere for 48 hours.

After the completion of the 48-hour stirring, 3 mL of pyridine solution was added for deprotection of the Fmoc moiety and conjugation of FA. The resulting mixture was stirred at 600 rpm for 30 minutes using a stirring device to synthesize DSPE-PEDS-FA conjugate.

The lipid-PEDS-FA conjugate synthesized was precipitated by addition to 100 mL of diethyl ether at −20° C., followed by centrifugation at 10,000 rpm for 15 minutes. Subsequently, the precipitate was subjected to vacuum drying at −80° C. and purified by dialysis (molecular weight cutoff 2 kD) in distilled water to remove impurities. The resulting material was then freeze-dried.

The chemical structure of the synthesized lipid-PEDS-FA conjugate was confirmed using NMR (500 MHz FT-NMR spectrometer, Bruker, Germany) and FT-IR (Perkin Elmer FTIR Spectrum Two, PerkinElmer, USA) spectroscopic analysis.

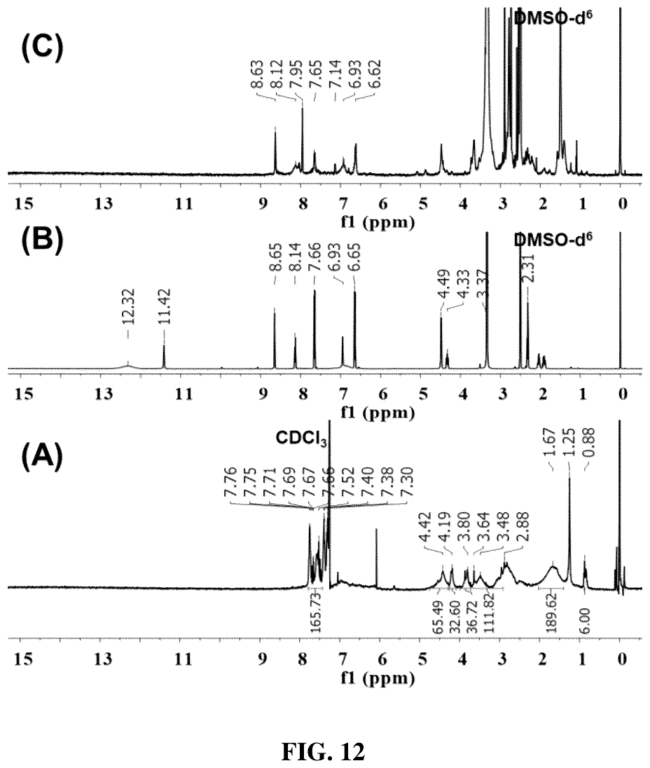

In FIG. 12, the chemical structure of succinoyl-lipid (DSPE)-Fmoc-PEDS is illustrated by (A), the chemical structure of folic acid (FA) is illustrated by (B), and the chemical structure of DSPE-lipid (PEDS)-FA is illustrated by (C), as confirmed through NMR spectra analysis.

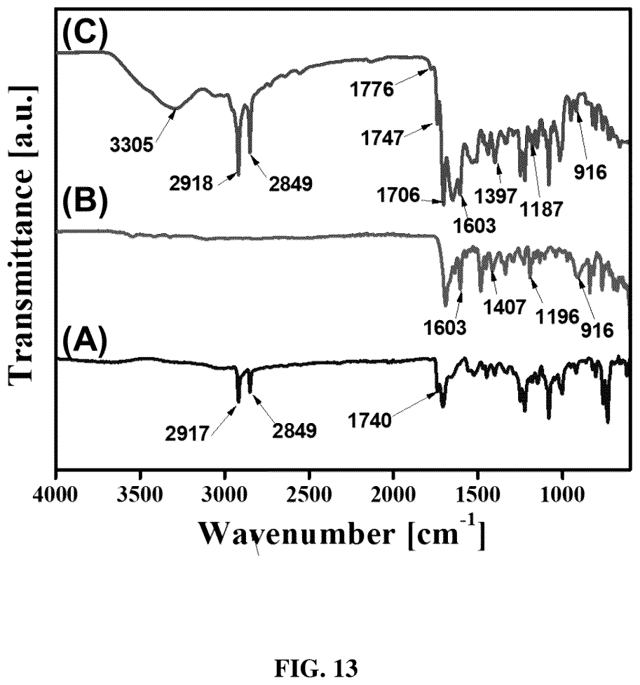

In FIG. 13, the chemical structure of succinoyl-lipid (PEDS)-Fmoc-PEDS is illustrated by (A), the chemical structure of folic acid (FA) is illustrated by (B), and the chemical structure of lipid (PEDS)-PEDS-FA is illustrated by (C), as verified through FT-IR spectra analysis.

### 1-4. Synthesis of DSPE-PEDS5CFL-Arg-PEG1K-FA Conjugate

21.23 μmol of 5-carboxyfluorescein (5CFL) and 106.15 μmol of Fmoc-Arg were dissolved in 5 mL of anhydrous dimethylformamide (DMF). To this solution, EDC was adjusted to a concentration of 1 mmol and NHS to a concentration of 1.5 mmol. The resulting solution was stirred at 25° C. for 1 hour to prepare the reaction mixture.

Subsequently, the DSPE-PEDS-FA conjugate from example 1-3 (with a concentration of grafted amine groups within the polymer of 212 μmol) and 20 μL of triethylamine were added to the stirred reaction mixture containing the 5CFL solution, Fmoc-Arg solution, EDC, and NHS dissolved in anhydrous dimethylformamide. The reaction mixture was stirred at 25° C. for 48 hours to prepare the reaction mixture.

Subsequently, the resulting reaction mixture was precipitated in 100 mL of diethyl ether at −20° C. for 48 hours, followed by dialysis (with a molecular weight cutoff of 2 kD) and freeze-drying to synthesize 100 mg of the compound DSPE-PEDS5CFL-Arg-PEG1k-FA, wherein 5CFL, PEG1k-NHS, Arginine, and DSPE-PEDS-FA are combined.

The functional moieties of the synthesized DSPE-PEDS_{5CFL-Arg-PEG1k}-FA conjugate were confirmed using NMR (500 MHz FT-NMR spectrometer, Bruker, Germany) and FT-IR (Perkin Elmer FTIR Spectrum Two, PerkinElmer, USA) spectroscopic analyses.

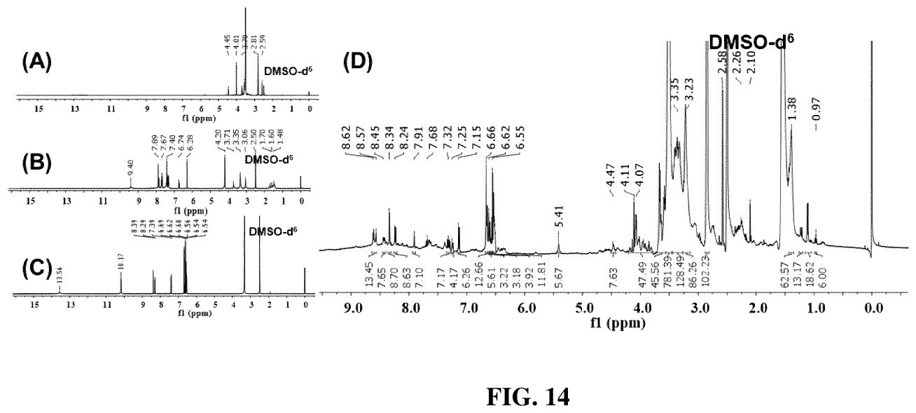

In FIG. 14, PEG1k-COOH is represented by (A), Fmoc-arginine is represented by (B), 5CFL dye is represented by (C), and the chemical structure of the synthesized multifunctional lipid-PEDS_{5CFL-Arg-PEG1k}-FA is represented by (D), as confirmed by NMR spectra.

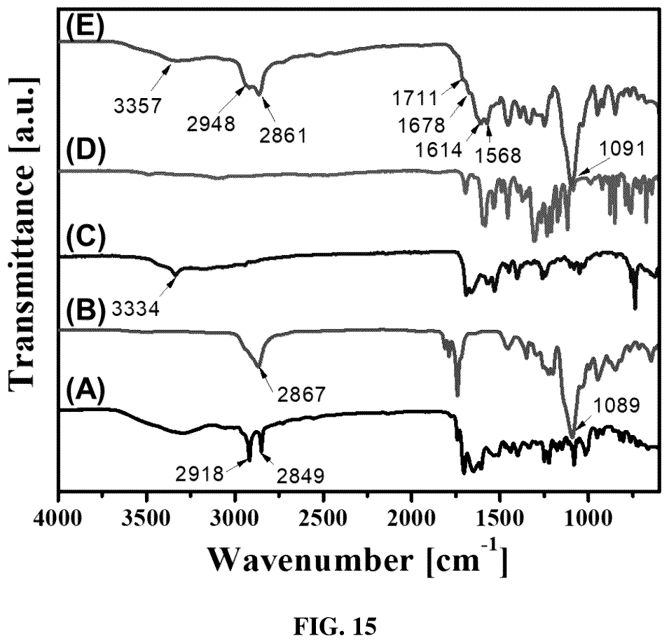

In FIG. 15, DSPE-PEDS-FA is represented by (A), PEG1k-COOH is represented by (B), Fmoc-arginine is represented by (C), the 5CFL dye is represented by (D), and the chemical structure of the synthesized multifunctional lipid-PEDS_{5CFL-Arg-PEG1k}-FA is represented by (E), as confirmed by FT-IR spectra.

### Example 2: Synthesis of DSPE-PEDS5CFL-Arg-PEG1k-Biotin

### 2-1. Synthesis of DSPE-PEDS-Biotin Conjugate

Using the DSPE-Fmoc-PEDS conjugate synthesized in manufacturing example 1-2, DSPE-Fmoc-PEDS-Biotin conjugate was prepared following the same procedure as example 1-3, with the substitution of 5.90 μmol of biotin for the 5.90 μmol of folic acid in the reaction mixture for conjugation with DSPE-Fmoc-PEDS.

The chemical structure of the synthesized succinoyl-DSPE-Fmoc-PEDS-Biotin conjugate was confirmed by NMR (500 MHZ FT-NMR spectrometer, Bruker, Germany) and FT-IR (Perkin Elmer FTIR Spectrum Two, PerkinElmer, USA) spectroscopic analysis.

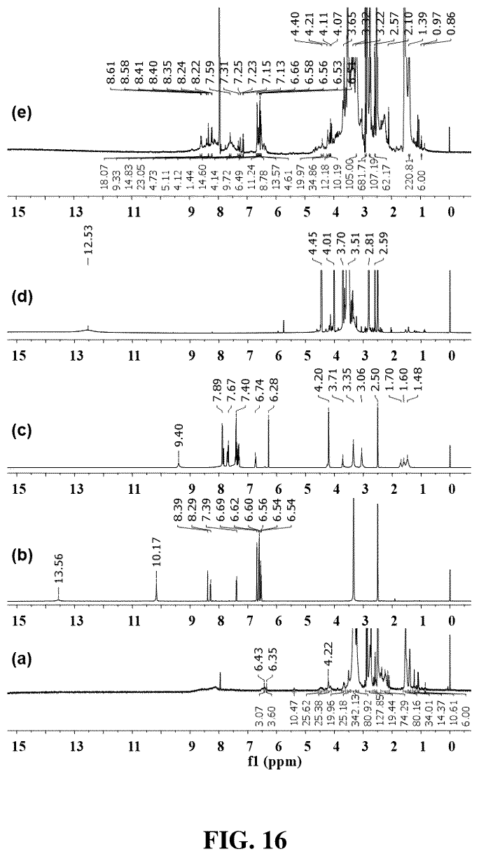

In FIG. 16, DSPE-PEDS-Biotin is represented by (a), 5-CFL is represented by (b), Fmoc-Arg is represented by (c), PEG1k is represented by (d), and the chemical structure of DSPE-PEDS_{5CFL/Arg/PEG1k}-Biotin as confirmed by NMR spectra is represented by (e).

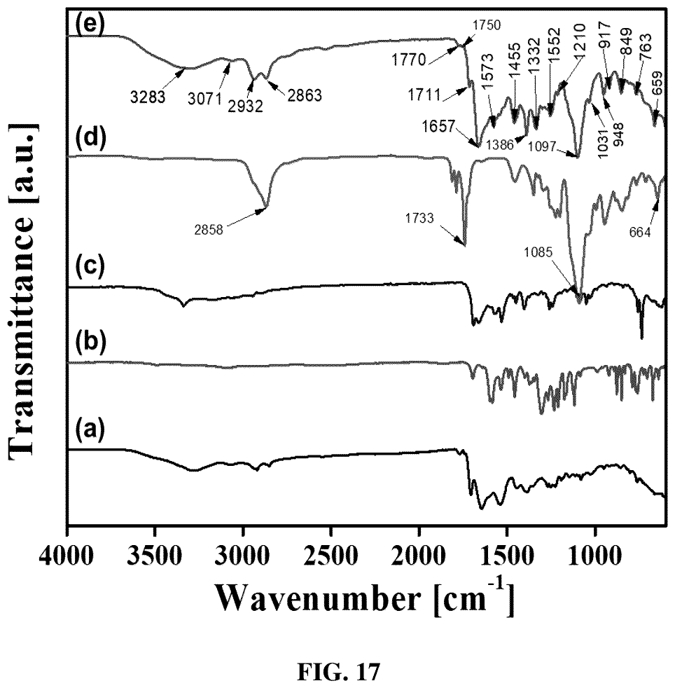

In FIG. 17, DSPE-PEDS-Biotin is represented by (a), 5-CFL is represented by (b), Fmoc-Arg is represented by (c), PEG1k is represented by (d), and the chemical structure of DSPE-PEDS_{5CFL/Arg/PEG1k}-Biotin as confirmed by FT-IR spectra is represented by (e).

### 2-2. Synthesis of DSPE-PEDS_{5CFL-Arg-PEG1k}-Biotin Conjugate

Using the DSPE-Fmoc-PEDS-Biotin conjugate synthesized in example 2-1 following the same method as example 1-4, a compound conjugated with 5CFL, PEG1k-NHS, and arginine to the lipid (DSPE)-PEDS-FA was synthesized to yield DSPE-PEDS_{5CFL-Arg-PEG1k}-Biotin conjugate.

The chemical structure confirmation of the functional moieties of the DSPE-PEDS_{5CFL-Arg-PEG1k}-Biotin conjugate was performed using NMR (500 MHz FT-NMR spectrometer, Bruker, Germany) and FT-IR (Perkin Elmer FTIR Spectrum Two, PerkinElmer, USA) spectroscopy.

In FIG. 16, the chemical structure of DSPE-PEDS-Biotin is represented by (a), the chemical structure of 5-CFL is represented by (b), the chemical structure of Fmoc-Arg is represented by (c), the chemical structure of PEG1k is represented by (d), and the chemical structure of DSPE-PEDS_{5CFL/Arg/PEG1k}-Biotin confirmed via NMR spectra is represented by (e).

In FIG. 17, the chemical structure of DSPE-PEDS-Biotin is represented by (a), the chemical structure of 5-CFL is represented by (b), the chemical structure of Fmoc-Arg is represented by (c), the chemical structure of PEG1k is represented by (d), and the chemical structure of DSPE-PEDS_{5CFL/Arg/PEG1k}-Biotin confirmed via FT-IR spectra is represented by (e).

### Example 3: Cell Culture

Natural killer cells binding to the surface of cancer cells were obtained by culturing NK-92 mi cells from the American Type Culture Collection (ATCC), USA.

The NK-92mi cells were seeded at a concentration of 1×10⁵ cells/mL in T25 culture flasks. The culture medium consisted of 12.5% fetal bovine serum (FBS, Gibco), 12.5% horse serum (Gibco), 1% penicillin-streptomycin solution (Corning, USA), 0.2 mM inositol (Sigma-Aldrich, USA), 0.1 mM β-mercaptoethanol (Sigma-Aldrich), and 0.02 mM folic acid (Sigma-Aldrich) in 10 mL of MEMα (Minimum Essential Medium Alpha, Gibco, USA). Cell culture was conducted for 48 hours at 37° C. under 5% CO2 and 95% humidity.

The MCF-7 (ATCC) breast cancer cell line, MDA-MB-231 (ATCC) breast cancer cell line, MIA PaCa-2 (ATCC) pancreatic cancer cell line, and normal human dermal fibroblast cell line (Lonza, USA) were also obtained from ATCC. These cells were cultured for 24 hours at 37° C. under 5% CO2 and 95% humidity in a culture medium consisting of 89% Dulbecco's modified Eagle's medium (DMEM, Corning), 1% penicillin-streptomycin solution, and 10% FBS (Corning).

The MCF-7 (ATCC) breast cancer cell line was seeded at a concentration of 5×10⁴ cells/mL in T25 culture flasks, while the MDA-MB-231 (ATCC) breast cancer cell line was seeded at the same concentration in T25 culture flasks. The MIA PaCa-2 (ATCC) pancreatic cancer cell line was also seeded at a concentration of 5×10⁴ cells/mL in T25 culture flasks. Additionally, normal human dermal fibroblast cells (Lonza, USA) were seeded at a concentration of 5×10⁴ cells/mL in T25 culture flasks for use in the experiments.

### Embodiment 1: Natural Killer Cells Cultured Under Coating Including DSPE-PEDS_{5CFL-Arg-PEG1k}-FA

NK-92mi cells cultured according to the method of Example 3, with a concentration of 5×10⁵ cells, were suspended in 10 mL of αMEM medium. Powder of DSPE-PEDS_{5CFL-Arg-PEG1k}-FA, prepared according to the method of Example 1, was dissolved in αMEM medium to obtain a coating solution with a concentration of 2 mg/mL. 100 μL of the coating solution was mixed with the NK-92mi cell suspension, and the mixture was cultured for 30 minutes at 25° C. to allow the surface of the NK-92mi cells to be coated with the DSPE-PEDS_{5CFL-Arg-PEG1k}-FA compound.

The coated NK-92mi cells were washed twice with 1 mL of DPBS (Dulbecco's phosphate-buffered saline, Sigma-Aldrich).

### Embodiment 2: Natural Killer Cells Cultured Under Coating Component Containing DSPE-PEDS_{5CFL-Arg-PEG1k}-Biotin Conjugate

A coating solution (2 mg/mL) was prepared by dissolving 1 mg of DSPE-PEDS5CFL-Arg-PEG1k-Biotin, synthesized according to Example 2, in 0.5 mL of αMEM at 100% concentration.

NK-92mi cells (5×10⁵ cells) were centrifuged to form a cell pellet. To this cell pellet, 0.1 mL of the aforementioned coating solution (2 mg/mL) was added and incubated at 25° C. for 30 minutes to prepare NK-92mi cells coated with DSPE-PEDS_{5CFL-Arg-PEG1k}-Biotin compound on the surface.

The surface-coated NK-92mi cells were washed twice with 1 mL of DPBS (Dulbecco's phosphate-buffered saline, Sigma-Aldrich).

### Comparative Example 1. Uncoated NK-92mi Cells

Cultured as in experimental examples 1 and 2, without the addition of DSPE-PEDS_{5CFL-Arg-PEG1k}-FA or DSPE-PEDS_{5CFL-Arg-PEG1k}-Biotin to the cell culture medium.

### Experimental Experiment 1. Confirmation of Coating Morphology, Coating Efficiency, and Coating Stability on the Surface of NK-92mi Cells

### 1-1: Confirmation of Coating Morphology

To visualize the coating morphology, the surface-coated NK-92mi cells were labeled with the fluorescent dye, 5-carboxyfluorescein, and examined using a fluorescence microscope (Ti-E System, Nikon, Japan).

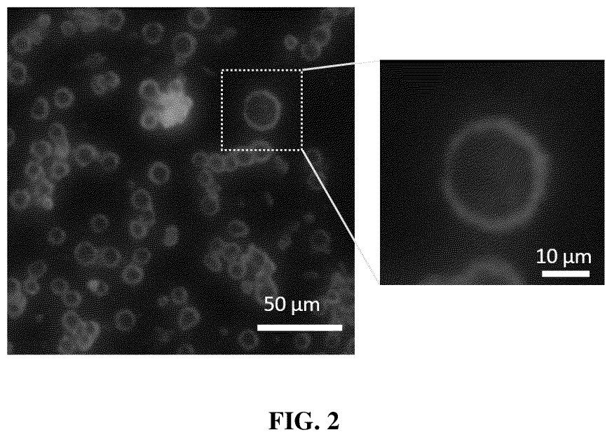

FIG. 2 is illustrating the successful coating of NK-92mi cell surfaces with the coating component containing DSPE-PEDS5CFL-Arg-PEG1k-FA, as confirmed by the results above.

### 1-2: Confirmation of the Efficiency of the Coating

The coating efficiency was determined by treating the surface of untreated NK-92mi cells with coating solutions of DSPE-PEDS_{5CFL-Arg-PEG1k}-FA at concentrations of 0.5 mg/mL, 1 mg/mL, and 2 mg/mL, respectively. Subsequently, the efficiency of cell coating was confirmed using a flow cytometer (Beckman Coulter, USA), and the optimal coating concentration of 2 mg/mL was determined.

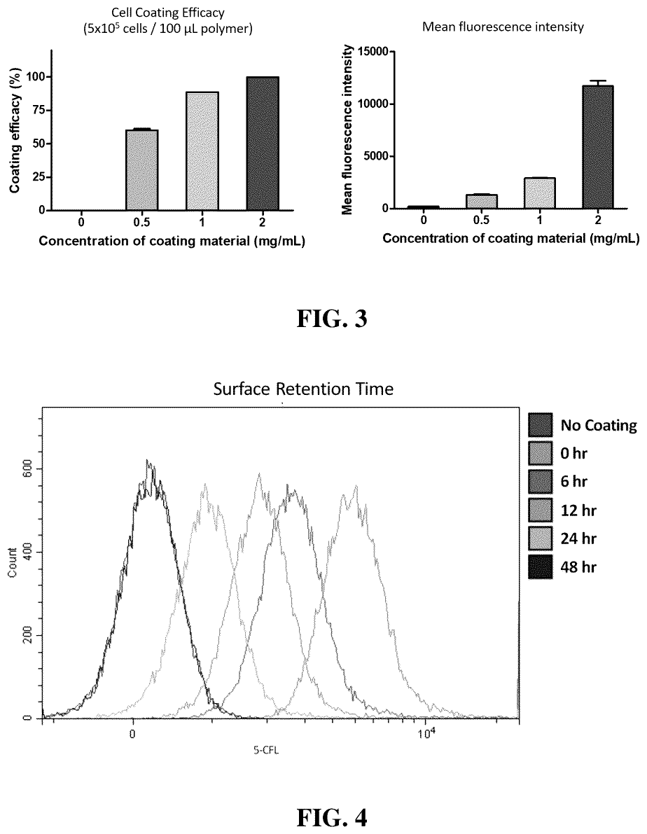

FIG. 3 is illustrating that on the left side, the change in coating efficiency of NK-92mi cells is depicted as the concentration of the coating solution varies (0.5 mg/mL, 1 mg/mL, and 2 mg/mL). On the right side, the coating efficiency of NK-92mi cells was analyzed using a fluorescent marker, and the fluorescence intensity of the supernatant was measured using microplate spectrophotometry (Ex/Em=485/535 nm wavelength) for each concentration.

Here, at a coating solution concentration of 2 mg/mL, the highest fluorescence intensity was measured, indicating that the coating efficiency at this concentration is the most superior.

### 1-3. Coating Stability Confirmation

The surface-coated NK-92mi cells were incubated in complete growth medium at 37° C.

Cells were collected at 0, 6, 12, 24, and 48 hours, and fluorescent signals were measured using a flow cytometer (Beckman Coulter, USA). This was compared to the fluorescent signal of non-coated NK-92mi cells to analyze the persistence of DSPE-PEDS_{5CFL-Arg-PEG1k}-FA conjugates until which time.

FIG. 4 is showing the fluorescent signal matches that of non-coated NK-92mi cells after 48 hours, indicating the presence of PEDS_{5CFL-Arg-PEG1k}-FA conjugates on the surface of NK-92 mi cells up to 48 hours post-coating.

### Experimental Experiment 2: Verification of Enhanced Cancer Cell Recognition Ability

MCF-7 breast cancer cells, MDA-MB-231 breast cancer cells, MIA PaCa-2 pancreatic cancer cells, and a control group of human fibroblast (hFibroblast) were seeded at a density of 10,000 cells per well in a 96-well plate and incubated at 37° C. with 5% CO2 for 24 hours.

Folic acid (1 mM) was added to the cell culture medium at a volume of 0.1 mL per well to saturate the folate receptors (FAR) on the surface of breast cancer cells and pancreatic cancer cells. The incubation was carried out at 37° C. for 2 hours.

NK-92mi cells were coated using the methods described in Example 1 or Example 2, and then labeled with 10 μM CellTracker™ Blue CMAC Dye (Invitrogen, USA) at 37° C. for 30 minutes. After labeling, the cells were washed twice with DPBS. As a control, NK-92mi cells from Comparative Example I were labeled with 10 μM CellTracker™ Blue CMAC Dye (Invitrogen, USA) under the same conditions and washed twice with DPBS.

The effector cells (NK-92mi cells from Comparative Example 1 or coated NK-92mi cells from Example 1 or 2) were co-cultured with target cells (MCF-7 breast cancer cells, MDA-MB-231 breast cancer cells, MIA PaCa-2 pancreatic cancer cells, and human fibroblast cells, respectively) at an effector-to-target (E: T) cell ratio of 10:1. The co-culture was conducted at 37° C. for 30 minutes, after which the unbound effector cells were collected. Subsequently, to calculate the remaining effector cells-to-target cells (E: T) ratio, the unbound effector cells were quantified using a standard curve based on the fluorescence intensity of labeled effector cells.

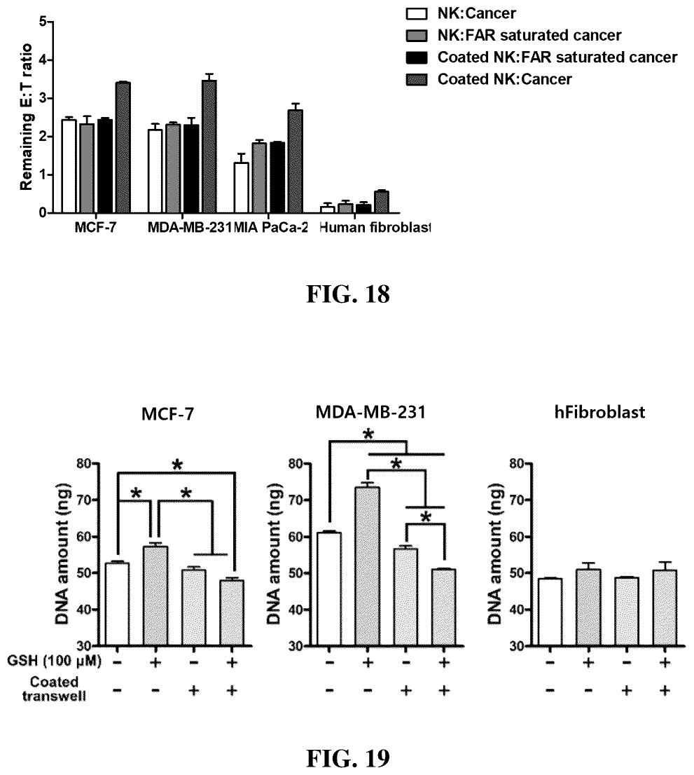

FIG. 18 is a graph showing the ratio of effector cells recognizing target cells relative to the target cells themselves.

Based on the results, it was observed that the coated NK-92mi cells exhibited the highest degree of recognition of cancer cells (MCF-7 breast cancer cells, MDA-MB-231 breast cancer cells, or MIA PaCa-2 pancreatic cancer cells) among the tested groups. This indicates that the cancer cell recognition ability of coated natural killer cells was significantly enhanced by approximately 1.4-fold compared to uncoated natural killer cells.

### Experimental Experiment 3: Confirmation of Enhanced Cancer Cell Cytotoxicity of Coated Natural Killer Cells-FA

The effector cells (NK-92mi cells, either untreated as in Control 1 or coated as in Embodiment 1 or 2) were co-cultured with target cells (MCF-7 breast cancer cells, MDA-MB-231 breast cancer cells, MIA PaCa-2 pancreatic cancer cells, and human fibroblast cells, respectively) at ratios of 1:1, 2:1, 4:1, and 10:1, respectively. The co-cultures were maintained at 37° C. with 5% CO2 and 95% humidity for 24 hours to verify the lysis or demise of MCF-7, MDA-MB-231, or MIA PaCa-2 cancer cells. Coated NK-92mi cells were prepared using the method described in Experiment 1, while untreated NK-92mi cells were cultured according to Control 1.

The confirmation of cell lysis/death by natural killer cells was conducted using the calcein-AM release assay. The quantification of cell lysis/death ratios was calculated using the disclosed calcein-AM release assay method.

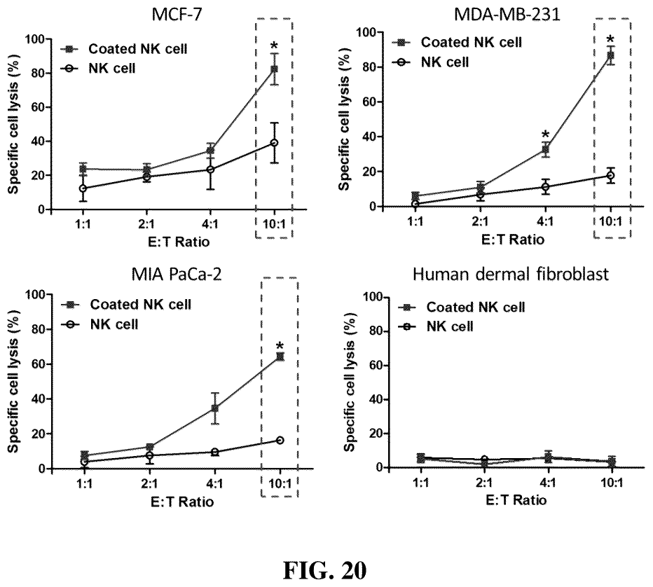

FIG. 20 is representing the cytotoxic effect of NK-92mi cells cultured according to the methods of Embodiment 1 and Comparative Example 1 on cancer cell lines.

As evident from FIG. 20, when NK-92mi cells coated with the DSPE-PEDS_{5CFL-Arg-PEG1k}-FA conjugate of Example 1 of the present invention were co-cultured with cancer cell lines, there was a significant increase in the cytotoxic effect on the cancer cell lines compared to co-culturing with uncoated NK-92mi cells. Moreover, the enhancement of the cytotoxic effect on cancer cell lines was observed to increase dramatically with the increasing number of NK-92mi cells coated with the DSPE-PEDS_{5CFL-Arg-PEG1k}-FA conjugate, indicating a synergistic effect with increasing coated NK-92mi cell numbers.

However, it was observed that even when co-cultured with human fibroblast cells instead of cancer cell lines as the control group, the induction of cell death was not observed regardless of the number of NK-92mi cells coated with the DSPE-PEDS_{5CFL-Arg-PEG1k}-FA conjugate of Example 1 of the present invention.

### Experimental Experiment 4: Confirmation of Enhanced Cancer Cell Killing Ability of Coated Natural Killer Cells-Biotin

Effecter cells (NK-92mi cells (Comparative Example 1) or coated NK-92mi cells (Embodiment 1 or 2)) were co-cultured with target cells (MCF-7 breast cancer cells, MDA-MB-231 breast cancer cells, MIA PaCa-2 pancreatic cancer cells, and human fibroblast cells) at ratios of 1:1, 2:1, 4:1, and 10:1, respectively, under conditions of 37° C., 5% CO2, and 95% humidity for 24 hours to verify the lysis or death of MCF-7 breast cancer cells, MDA-MB-231 breast cancer cells, or MIA PaCa-2 pancreatic cancer cells. Coated NK-92mi cells were prepared using the method described in embodiment 2, while uncoated NK-92mi cells were prepared using the method described in Comparative Example 1.

Cell lysis/death induced by natural killer cells was assessed using the calcein-AM release assay. The quantitative ratio of cell lysis/death was calculated using the established calcein-AM release assay method.

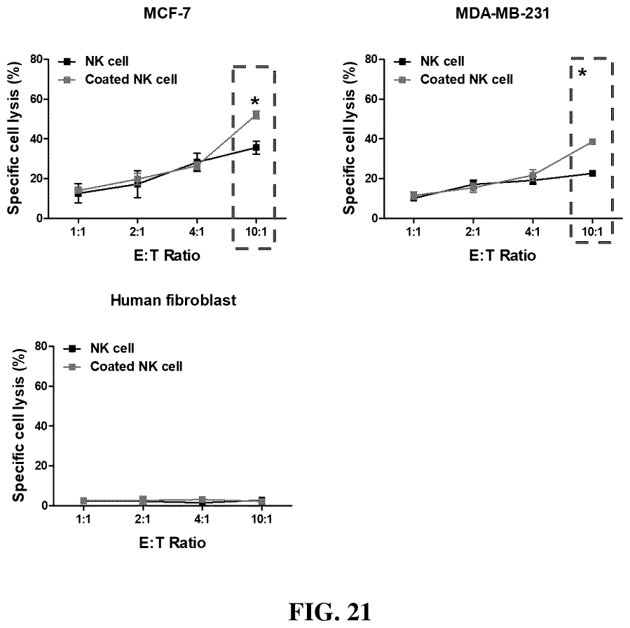

FIG. 21 is illustrating the cytolytic effects of NK-92mi cells cultured according to Embodiment 2 and Comparative Example 1 methods on cancer cell lines.

The results from FIG. 21 demonstrate a significant enhancement in the cytolytic effects of cancer cell lines when co-cultured with natural killer cells coated with DSPE-PEDS5CFL-Arg-PEG1k-Biotin conjugate prepared according to Example 1 of the present invention, compared to the co-culture with uncoated natural killer cells. Moreover, by gradually increasing the number of natural killer cells coated with DSPE-PEDS5CFL-Arg-PEG1k-Biotin conjugate prepared according to Example 2 of the present invention, the promotion of cancer cell death becomes more pronounced with increasing co-culture, as evidenced by FIG. 21.

However, it was observed that even when co-cultured with human fibroblast cells instead of cancer cell lines, the induction of cell death was not observed, regardless of the increased number of natural killer cells coated with DSPE-PEDS_{5CFL-Arg-PEG1k}-Biotin conjugate prepared according to Example 2 of the present invention.

### Experimental Experiment 5: Confirmation of Cancer Cell Death Effect Through Glutathione Release Detection

Following the same procedure as Experimental Experiment 3, the effector cells (NK-92 mi cells from Comparative Example 1 or coated NK-92mi cells from Embodiment 1 or 2) were co-cultured with the target cells (MCF-7 breast cancer cells, MDA-MB-231 breast cancer cells, MIA PaCa-2 pancreatic cancer cells, and human fibroblast cells) at a ratio of 10:1. The co-culture was incubated at 37° C. with 5% CO2 and 95% humidity for 4 hours to measure the extent of glutathione (GSH) release from MCF-7 breast cancer cells, MDA-MB-231 breast cancer cells, or MIA PaCa-2 pancreatic cancer cells. Triton X-100 was used as a positive control to artificially disrupt cancer cells and induce the release of intracellular glutathione (GSH).

The measurement of glutathione released from disrupted cancer cells was performed using the EZ-glutathione assay kit (DoGenBio) according to the manufacturer's protocol.

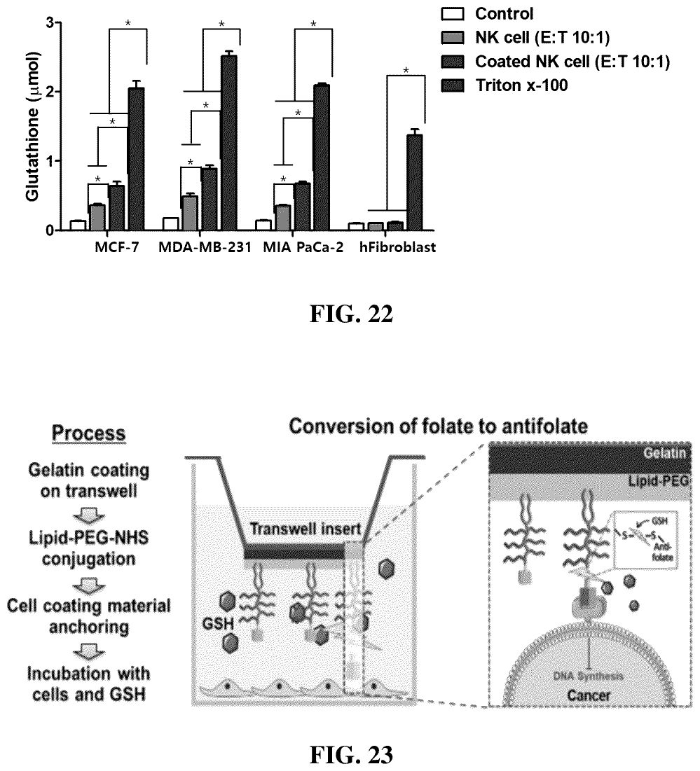

FIG. 22 is a graph showing the concentration of glutathione released from each target cell (MCF-7 breast cancer cells, MDA-MB-231 breast cancer cells, MIA PaCa-2 pancreatic cancer cells, or human fibroblast cells).

The results from FIG. 22 demonstrate that when NK-92mi cells coated with the DSPE-PEDS5CFL-Arg-PEG1k-Biotin conjugate of Example 1 are co-cultured with cancer cells, there is a significantly enhanced glutathione release compared to co-culturing with uncoated NK-92mi cells. This indicates a remarkable improvement in the glutathione release effect when using NK-92 mi cells coated with the DSPE-PEDS5CFL-Arg-PEG1k-Biotin conjugate of Example 1. Thus, it can be concluded that the coated NK-92mi cells of Example 1 exhibit superior efficacy in destroying cancer cells.

In the co-culture of natural killer cells and cancer cells, the natural killer cells destroyed the cancer cells, leading to the release of glutathione (GSH). This effect was more pronounced in the coated NK cell group, resulting in increased induction of cancer cell death and subsequently higher levels of glutathione release.

As seen in FIG. 22, it is evident that even in the control group treated with Triton-X100, representing non-cancerous cell lines, glutathione (GSH) release is observed upon cell death.

However, when natural killer cells or coated natural killer cells were co-cultured with normal cells, there was no release of glutathione (GSH) observed, indicating that the natural killer cells, or coated natural killer cells, did not induce destruction of the normal cells.

### Experimental Experiment 6: Induction of Cancer Cell Death Via Antifolate Mediation

To confirm the effect of anticancer cell death mediated by anti-folate, a structure was fabricated by immobilizing the DSPE-PEDS_{5CFL-Arg-PEG1k}-FA conjugate of the present invention on gelatin-coated solution. Subsequently, experiments were conducted to mimic the glutathione environment emanating from cancer cells as follows.

10 μL of 2% (w/v) gelatin solution (porcine skin-derived, Sigma-Aldrich, G1890) was dropped onto the bottom surface of a polycarbonate transwell (pore size 0.4 μm, Corning, CLS3428-24EA), and the gelatin-treated transwell was dried at room temperature for 2 hours.

Subsequently, 1 mL of a 5 mM solution of 1,2-distearoyl-sn-glycero-3-phosphoethanolamine-N-[succinimidyl (PEG)] (DSPE-PEG-NHS, Nanosoft Polymers, USA) was added to the gelatin-coated transwell, and the mixture was stirred for 1 hour to allow conjugation between the amino groups of gelatin and the NHS moiety of 1,2-distearoyl-sn-glycero-3-phosphoethanolamine-N-[succinimidyl (PEG)], exposing the lipid portion (DSPE) outward.

Lastly, a coating solution of 2 mg/mL, as prepared in Embodiment 1, was applied to the aforementioned lipid for 30 minutes to manufacture a structure mimicking the surface of natural killer cells (NK cells) (FIG. 23).

As in Experimental example 1, MCF-7 breast cancer cells, MDA-MB-231 breast cancer cells, MIA PaCa-2 pancreatic cancer cells, and normal human fibroblast (hFibroblast) cells were seeded in 24-well plates at a density of 10,000 cells per well and incubated at 37° C. with 5% CO2 and 95% humidity for 24 hours.

The cells were then treated with trypsin to detach them, followed by centrifugation to collect them. The collected cells were lysed using a homogenizer, and the amount of DNA from the target cells cultured in the transwell was quantified using the Quant-iT PicoGreen dsDNA Reagent Kit (Thermo Fisher Scientific, USA) according to the manufacturer's protocol.

FIG. 19 is illustrating whether the disulfide linkage of DSPE-PEDS_{5CFL-Arg-PEG1k}-FA, as per Example 1 of the present invention, is cleaved by glutathione, leading to the release of FA, which interferes with DNA synthesis in cancer cells and promotes cancer cell death.

based on the results, it was confirmed that the disulfide linkage of DSPE-PEDS5CFL-Arg-PEG1k-FA, as per Example 1 of the present invention, is cleaved by glutathione (GSH) treatment in the environment of gelatin-coated Transwell, leading to the release of folate (FA). Subsequently, after the release, the folate moiety, which is the cancer cell recognition moiety, is converted into an antifolate, thereby inducing further cancer cell death.

### Experimental Experiment 7: Measurement of Coated Natural Killer Cell Survival Capacity

The NK-92mi cells cultured using the method of Example 3 were mixed with a coating solution prepared by dissolving 100 μL of 2 mg/mL powder of DSPE-PEDS5CFL-Arg-PEG1k-FA, prepared according to the method of Example 1, in 10 mL of αMEM medium. The mixture was then cultured with the NK-92mi cells at 25° C. for 30 minutes to prepare the surface-coated NK-92mi cells with the DSPE-PEDS5CFL-Arg-PEG1k-FA compound.

During the preparation process of the surface-coated NK-92mi cells mentioned above, NK-92mi cells were separately prepared with coating solution concentrations of 0.5 mg/mL and 1 mg/mL.

The surface-coated NK-92mi cells, prepared at each adjusted concentration (0.5 mg/mL, 1 mg/mL, 2 mg/mL), were washed three times with 1 mL of DPBS (Dulbecco's phosphate-buffered saline, Sigma-Aldrich).

The surface-coated NK-92mi cells, adjusted to each washed concentration as mentioned above, were assessed for their viability using the WST-1 assay with EZ-Cytox (DoGenBio, Korea). The absorbance at 450 nm was measured using the Infinite M200 microplate reader (Tecan, Zurich, Switzerland).

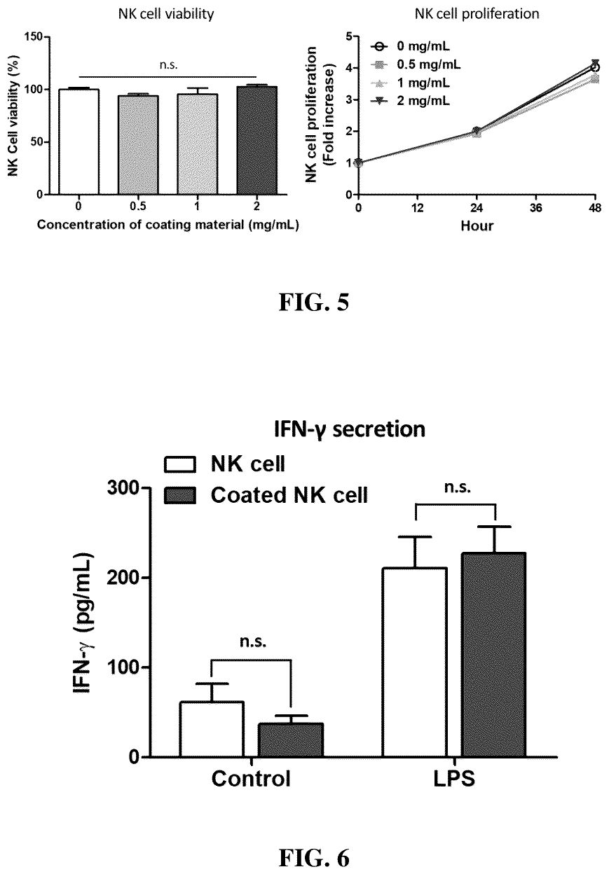

The left graph in FIG. 5 represents the results of measuring the viability of surface-coated NK-92mi cells at each adjusted concentration using the WST-1 assay. When compared to the untreated control group, there was minimal change in absorbance, indicating that coating the surface of NK-92mi cells with solutions containing DSPE-PEDS_{5CFL-Arg-PEG1k}-FA compounds up to a concentration of 2 mg/mL had no effect on cell viability.

### Experimental Experiment 8: Assessment of Proliferative Capacity of Coated Natural Killer Cells

NK-92mi cells cultured according to the method described in Example 3 were used. A powder of DSPE-PEDS_{5CFL-Arg-PEG1k}-FA prepared according to Example 1 was dissolved in αMEM medium to create a coating solution with a concentration of 2 mg/mL. 100 μL of this solution was mixed with 10 mL of αMEM medium containing the cultured NK-92mi cells. The mixture was then incubated at 25° C. for 30 minutes to prepare NK-92mi cells coated with the DSPE-PEDS_{5CFL-Arg-PEG1k}-FA compound.

In the preparation process of the surface-coated NK-92mi cells described above, separate preparations of NK-92mi cells were made with coating solution concentrations adjusted to 0.5 mg/mL and 1 mg/mL, respectively.

The surface-coated NK-92mi cells prepared at each concentration (0.5 mg/mL, 1 mg/mL, 2 mg/mL) were washed three times with 1 mL of DPBS (Dulbecco's Phosphate Buffered Saline, Sigma-Aldrich).

Long-term cell culture was conducted to compare the proliferation ability according to cell culture density. The proliferation ability of each cell was assessed by calculating the proliferation rate for each generation based on the initial inoculum cell count and the number of cells obtained after culture. The results are shown in the right graph of FIG. 6.

### Experimental Experiment 9: Measurement of Immune Factor Secretion by Coated Natural Killer Cells

In order to ascertain the extent of immune factor secretion via antigen recognition facilitated by the coated NK-92mi cells prepared according to Embodiment 1, the analysis focused on the secretion quantity of interferon-gamma (IFN-γ), which is reliant on lipopolysaccharide (LPS).

1.5×10⁵ of uncoated NK-92mi cells from Comparative Example 1 and 1.5×10⁵ of uncoated NK-92mi cells from Example 1 were cultured in media treated with 1 μg/mL of LPS (Escherichia coli O26: B6, Sigma-Aldrich). The cells were then incubated at 37° C. for 24 hours and the media collected after centrifugation.

Subsequently, 1.5×10⁵ uncoated NK-92mi cells from Comparative Example 1 and 1.5×10⁵ uncoated NK-92mi cells from Embodiment I were each incubated at 37° C. for 24 hours and the media collected after centrifugation for use as controls.

As observed in FIG. 6, in the untreated control group, the interferon-gamma secretion of uncoated NK-92mi cells was slightly higher compared to that of coated NK-92mi cells. However, in the experimental group treated with lipopolysaccharide, the secretion of interferon-gamma by coated NK-92mi cells was higher than that of uncoated NK-92mi cells.

Thus, it was observed that the interferon-gamma secretion of NK-92mi cells was comparable to that of coated NK-92mi cells, and in the experimental group treated with lipopolysaccharide, the interferon-gamma secretion of NK-92mi cells was enhanced to a level similar to that of coated NK-92mi cells.

In summary, it was confirmed that the coating material on the surface of NK cells did not affect the series of processes where cell signaling occurs following the recognition of antigenic substances such as LPS, leading to the release of interferon-gamma into the extracellular environment.

### Experimental Experiment 10: Confirmation of the Antitumor Effect of Coated Natural Killer Cells In Vivo

Preparation of the MDA-MB-231 Xenograft model for confirmation of the antitumor effect of coated NK-92mi Cells, following the method described in Embodiment 1.

The xenograft model was prepared by injecting 1×10⁷ MDA-MB-231 cells subcutaneously into the left flank of 40 female BALB/c nu/nu mice (Narabio, Korea) at 8 weeks of age.

Subsequently, when the tumor volume of the xenograft mice reached 100 mm³, the mice were randomly categorized into control group, atezolizumab injection group, uncoated NK-92mi cell injection group, and coated NK cell injection group (n=10 per group).

The categorized control group or each injection group was administered with 250 μL of phosphate-buffered saline (PBS).

Subsequently, the group injected with uncoated NK-92mi cells received an intravenous injection of 1×10⁷ uncoated NK-92mi cells, while the group injected with coated NK cells received an intravenous injection of 1×10⁷ coated NK cells. Meanwhile, the Atezolizumab injection group received intraperitoneal injections of Atezolizumab (BioXCell, USA) at a dosage of 10 mg/kg every 3 days.

The control group received only an injection of 250 μL of phosphate-buffered saline (PBS) during the aforementioned procedure, without any other injections.

Subsequently, 5 mice per group (total of 20 mice) were randomly selected for the analysis of tumor volume changes, tumor growth inhibition (TGI) rate changes, multiplicative changes in body weight, and survival probability, as described in the following experimental examples.

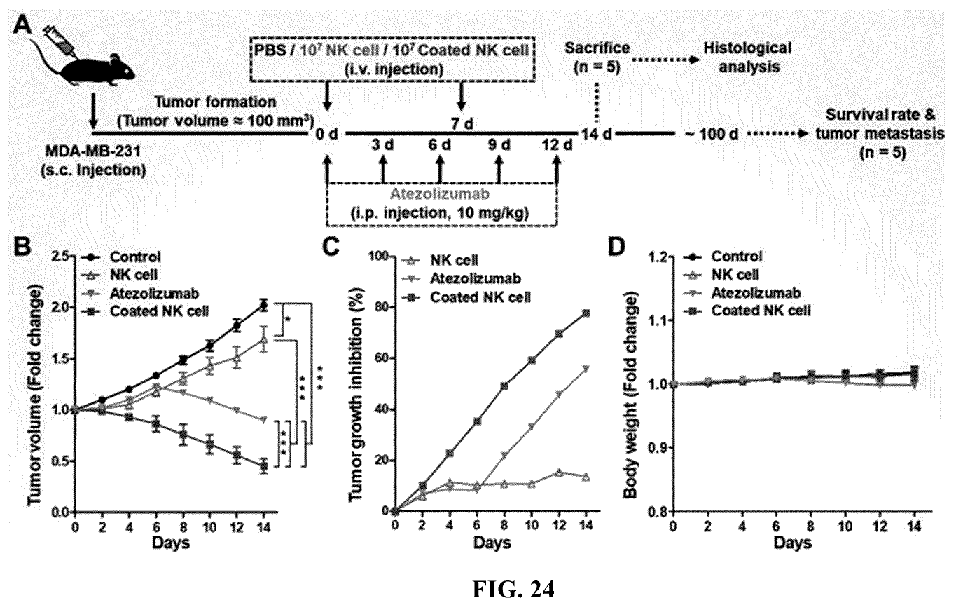

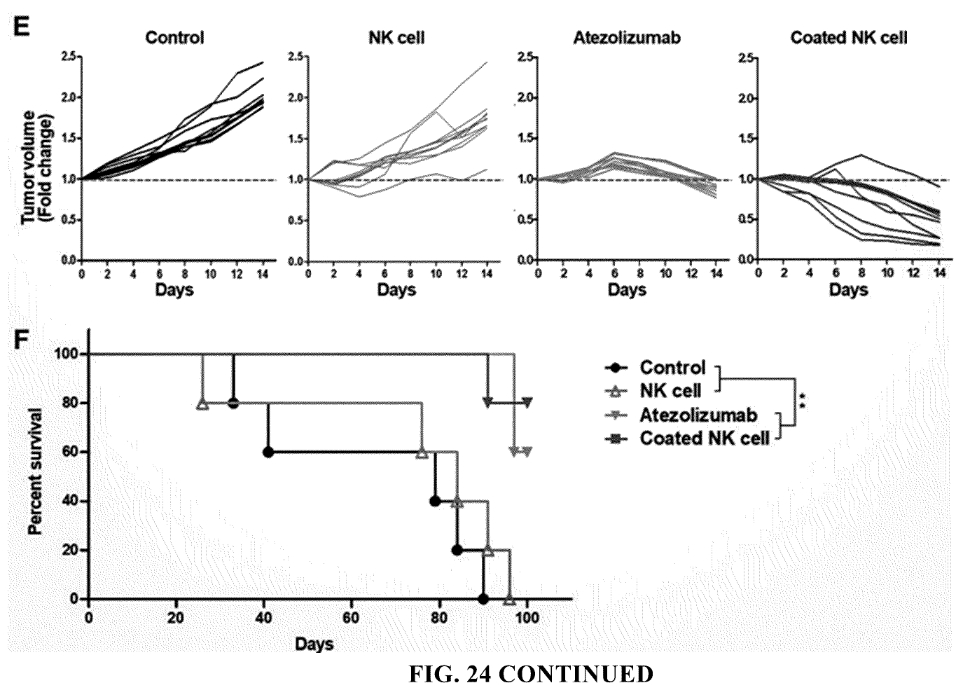

The process for confirming the in vivo tumor cell killing effect of coated natural killer cells is schematically outlined in FIG. 24 (A), depicting the experimental setup.

### 10-1: Measurement of Tumor Volume Change

To investigate the effect of cancer cell eradication in vivo, tumor growth was monitored for 14 days, and the recorded values were divided by the initial values to calculate the tumor volume according to the following formula:

Tumor Volume=0.5×a×b² (Where a represents the longest diameter of the tumor tissue and b represents the shortest diameter of the tumor tissue).

As depicted in FIG. 24 (E), the tumor volumes of the mice in the group injected with coated NK-92mi cells were lower compared to those in the other control or injection groups.

Additionally, as shown in FIG. 24 (B), the average tumor volume in the group injected with uncoated NK-92mi cells was suppressed compared to the control group; however, tumors continued to grow steadily. In contrast, the group injected with coated NK-92mi cells exhibited a noticeable decrease in average tumor volume from day 4 onwards. By the end of 14 days, the tumor volume in the coated NK-92mi cell injection group was one-fourth of that in the control group and half of that in the Atezolizumab injection group, indicating a significant effect on tumor cell eradication.

### 10-2: Measurement of the Tumor Growth Inhibition Ration

To assess the anti-tumor efficacy within the body, tumor growth was monitored for 14 days, and the tumor growth inhibition ratio (TGI) was calculated using the following formula:

$$TGI ( \% ) = 100 - ( \frac{V / {V}_{0}}{{V}_{k} / {V}_{k0}} \times 100 )$$

(Here, V represents the average tumor volume of the treated group at a specific time point, V0 denotes the initial average tumor volume of the treated group, VK indicates the average tumor volume of the control group at the same time point, and VK0 represents the initial average tumor volume of the control group).

Here, the average tumor volume refers to the mean value obtained by calculating the tumor volume for each mouse using the formula provided in Experiment 10-1, and then averaging these values.

The tumor growth inhibition ratio was determined as follows: in the untreated NK-92mi cell injection group, it was 10%, whereas in the coated NK-92mi cell injection group, it was 80%. This indicates a significant anti-tumor effect in the coated NK-92mi cell injection group, with an inhibition ratio 8 times higher than that of the untreated NK-92mi cell injection group, as shown in C of FIG. 24.

Meanwhile, in the Atezolizumab injection group, tumor inhibition was significantly enhanced compared to the control group and the untreated NK-92mi cell injection group. However, when compared to the coated NK-92mi cell injection group, at the 14-day mark, tumor volume was twice as large, and the tumor growth inhibition rate was 60%. This indicates a relatively diminished anti-cancer effect compared to the coated NK-92mi cell injection group.

### 10-3: Measurement of Change in Body Weight

To assess the effect of cancer cell eradication in vivo, the average body weight of mice in each control and treatment group was monitored over a period of 14 days.

As depicted in D of FIG. 24, the relative change in body weight remained relatively stable across mice classified into each group, indicating that the administered treatments did not induce systemic toxicity.

### Experimental Experiment 10-4: Measurement of Survival Probability

To investigate the effect of cancer cell elimination in vivo, the survival probability of mice categorized into each group (10 mice per group) was monitored for 100 days.

$$Survival probability = \frac{the \cdot daily \cdot survival \cdot count}{total \cdot number \cdot of \cdot individuals \cdot in \cdot each \cdot group}$$

(Here, daily survival count refers to the number of individuals observed to be surviving at a specific time point during the day.)

The survival probabilities at the end of 100 days were 80% for the coated NK-92mi cell injection group, 60% for the Atezolizumab injection group, and 0% for both the control group and the non-coated NK-92mi cell injection group, as shown in FIG. 24F. Notably, the coated NK-92mi cell injection group exhibited significantly superior anticancer efficacy compared to the control group and other injection groups.

### Experimental Experiment 11: Enhanced Anticancer Efficacy and Biocompatibility Confirmation of Coated Natural Killer Cells Through Histological Analysis

The same method as Experimental experiment 10 was used to create the xenograft mouse model. Then, 5 mice from each group (total of 20 mice) were randomly selected and euthanized for histological analysis.

Tumors and major organs collected from the xenograft mice in each group were fixed in phosphate-buffered saline (PBS) containing 4% paraformaldehyde for 24 hours and then stored in paraffin.

Subsequently, the tissue sections (5-μm thickness) were stained with fluorescent dyes. The slides were heated in 10 mM citrate buffer (pH 6.0) for 20 minutes at 98° C. and then cooled for 20 minutes at 25° C.

Following the cooling step, the specimens were incubated at 4° C. for 16 hours in solutions containing antibodies recognizing CD56 (MAB24083; R&D Systems, USA; 1:200), cleaved caspase-3 (9661, Cell Signaling Technology, USA; 1:200), Ki67 (ab16667, Abcam; 1:200), HMGB-1 (ab18256, Abcam; 1:100), CD8a (14-0081-82, Invitrogen; 1:100), HLA (Class I ABC, ab70328, Abcam; 1:200), and von Willebrand factor (vWF, AB7356, Sigma-Aldrich; 1:200).

Following the incubation, the specimens were incubated at 20° C. for 2 hours with secondary antibodies (Invitrogen; 1:200) and counterstained with TO-PRO-3 (TP3, T3605, Invitrogen, 1 μM).

Fluorescence detection was performed using a confocal laser microscope (DMi8; Leica, Germany). Tumor and necrotic areas were tracked from the H&E images, and measurements were made using ImageJ software. The number of marker-positive cells was counted, and specific marker-positive areas were measured from the immunohistochemical images. Five regions of interest (ROIs; 200×200 μm) were randomly selected from each single-stained slide of the samples (total of 25 ROIs per group).

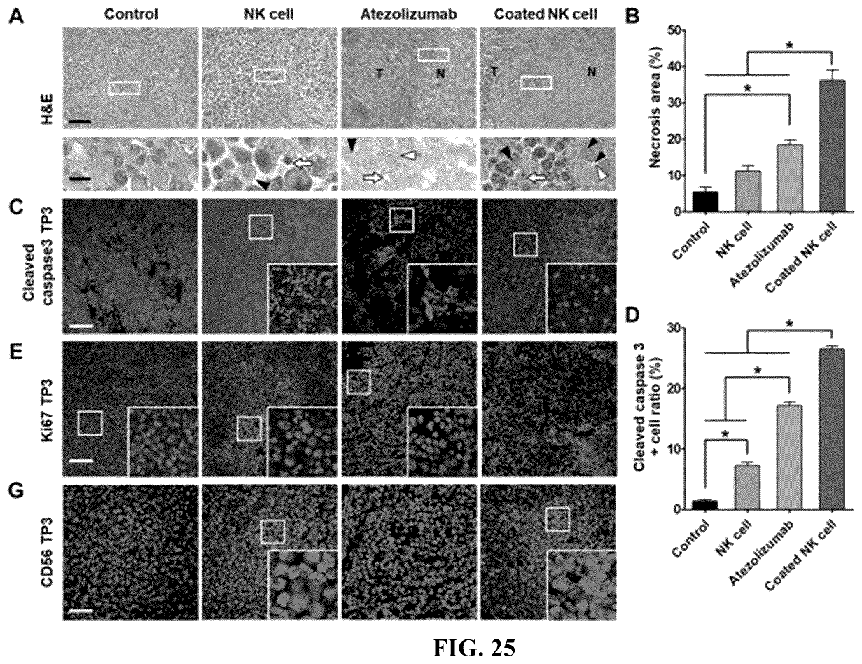

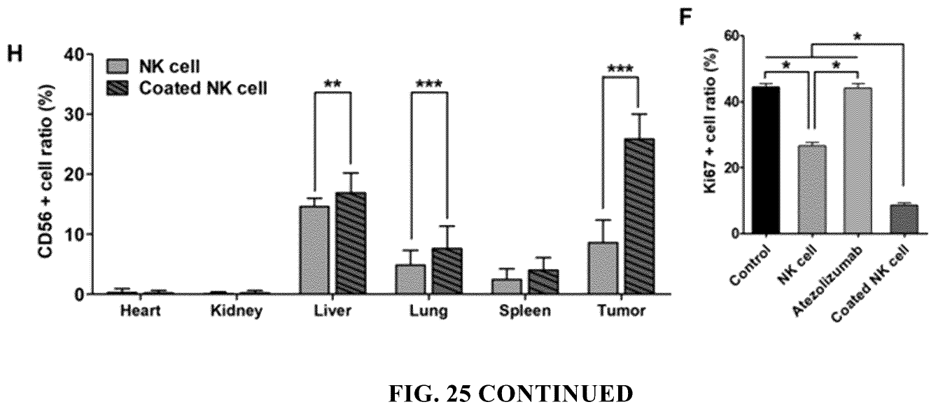

Tumor tissues from MDA-MB-231 xenograft mice (five mice per group) were stained with hematoxylin and eosin (H&E). In A of FIG. 25, T represents the tumor tissue area, and N represents the necrotic area.

The tumor tissues stained with hematoxylin and eosin (H&E) showed, as depicted in FIG. 25A, a higher presence of necrotic features such as eosinophilic cytoplasmic swelling, nuclear pyknosis, and karyorrhexis in the atezolizumab and coated NK-92mi cell injection groups compared to the control and untreated NK-92mi cell injection groups. This observation suggests a pronounced anti-tumor effect in the NK-92mi cell injection groups.

Additionally, as shown in FIG. 25B, the percentage of necrotic area was highest in the coated NK-92mi cell group at 36%, indicating the most extensive tumor necrosis induction compared to other groups.

The cleaved caspase-3 protein, detected in the tumor tissues, induces apoptosis in cancer cells. Higher expression of cleaved caspase-3 indicates more active cell death induction, suggesting superior anticancer efficacy.

The analysis of the expression level of cleaved caspase-3 revealed that, as shown in C of FIG. 25, significantly higher expression was observed in the Atezolizumab and coated NK-92mi cell injection groups compared to the control group and the group injected with uncoated NK-92 mi cells. This indicates a pronounced anticancer effect in the NK-92mi cell injection groups.

E and F of FIG. 25 depict the extent of tumor cell proliferation using the proliferation marker Ki67. Here, widespread expression of Ki67 was observed in the control group, Atezolizumab injection group, and uncoated NK-92mi cell injection group. However, significantly lower expression was observed in the coated NK-92mi cell injection group.

Human-specific CD56 (natural killer cell marker) was used to determine the relative ratio of tumor or infiltrated cells in the groups injected with coated NK-92mi cells compared to those injected with uncoated NK-92mi cells.

The results, as shown in G of FIG. 25, indicate a significant increase in the number of human-specific CD56-expressing NK cells in the samples injected with coated NK-92mi cells compared to those injected with uncoated NK-92mi cells. This suggests that coating NK cells with the polymer compound described in Embodiment 1 of the present invention enhances their ability to infiltrate cancer cells compared to uncoated NK cells, thereby demonstrating superior cancer cell eradication efficacy.

The relative proportion of tumor or infiltrated cells was found to be lower in the samples injected with coated NK-92mi cells.

In H of FIG. 25, the CD56+ cell ratio represents the relative proportion of human-specific CD56 expressed in total cells. Here, the relative ratio of CD56 was observed in the liver, lung, and spleen, but not in the heart and kidney.

In conclusion, histological analysis revealed that the administration of coated natural killer cells effectively inhibited tumor growth in the xenograft model.

The embodiments described herein are illustrative and not exhaustive. Various modifications and adaptations will be apparent to those skilled in the relevant arts without departing from the scope of the disclosure, as defined by the appended claims. For instance, the order of steps described may be altered, and components may be combined or substituted with equivalent elements to achieve similar outcomes. Therefore, alternative implementations, examples, and equivalents falling within the scope of the claims are considered to be part of the disclosure.

### MODE FOR INVENTION

Below, the present invention is described in detail.

The natural killer cell (NK cell) is a subset of lymphocytes involved in non-specific immunity. NK cells can be obtained through various techniques known in the art, including blood sample collection, cell component isolation methods, and the like.

The characteristics and biological properties of natural killer cells include the expression of surface antigens such as CD16, CD56, and/or CD57; the absence of alpha/beta or gamma/delta TCR complexes on the cell surface; the ability to bind to cells that fail to express “self” MHC/HLA antigens due to activation of specific cytolytic enzymes and kill them; the ability to kill tumor cells or cells from other diseases expressing NK activation receptor ligands; the ability to release cytokines that stimulate or inhibit immune responses; and the ability to undergo multiple rounds of cell division and produce daughter cells with biological properties similar to parent cells.

In the present invention, specific details related to the technical features and effects of the polymer compound may be applied similarly or analogously to pharmaceutical compositions, methods for the prevention or treatment of cancer, manufacturing methods of the polymer compound, and the inclusion or utilization of the polymer compound, unless they depart from the essence of the present invention.

The present invention provides a polymer compound comprising a minority moiety that binds to immune cells, a cancer cell recognition moiety, and a linker comprising the structure of Chemical Formula 1.

According to embodiments of the present invention, the immune cells may be T cells, B cells, or natural killer cells, and preferably, natural killer cells, although this is not limiting.

Specifically, the polymer compound of the present invention may provide a polymer compound comprising a minority moiety that binds to natural killer cells, a cancer cell recognition moiety, and a linker comprising the structure of Chemical Formula 1.

In the present invention, “surface modification” may refer to the attachment of a hydrophobic moiety to the surface of natural killer cells.

At one end of the chemical formula 1, the hydrophobic moiety can be attached, while at the other end of the chemical formula 1, the cancer cell targeting moiety can be attached, wherein n may be an integer ranging from 30 to 50.

At one end of the chemical formula 1, the hydrophobic moiety can be connected or linked through an amide bond, while at the other end of the chemical formula 1, the cancer cell targeting moiety can also be connected or linked through an amide bond.

The term “connection” as used herein may refer to the linking of one end of chemical formula 1 to the hydrophobic moiety. This linkage may entail a direct connection between one end of chemical formula 1 and the hydrophobic moiety, or it may involve an indirect connection. In instances of indirect connection, it signifies that one end of chemical formula 1 and the hydrophobic moiety are joined via a linker or similar structure. The term “connection” encompasses a chemical bond.

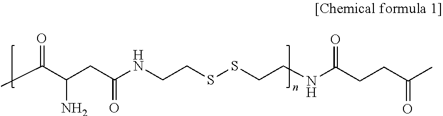

If n is less than 30, there may be a problem where compounds for preventing cellular internalization, cationic amino acids, or fluorescent dye compounds do not adequately bind within the structural unit. If n exceeds 50, the length of the chain may become excessively long, resulting in too many disulfide bonds within the structural unit, which may prevent proper connection between natural killer cells and cancer cells.

The cleavage of the disulfide bonds can occur randomly within the n repeating units of chemical formula 1, resulting in the formation of fragments of various lengths. However, it has been confirmed that this does not affect the efficacy of the cancer cell recognition moiety, which binds to the other end of chemical formula 1 and recognizes cancer cells for their destruction, nor does it induce cellular cytotoxicity.

The hydrophobic moiety binds to natural killer cells, while the cancer cell recognition moiety can recognize cancer cells and promote their destruction.

In embodiment of the present invention, the end of the chemical formula 1 may include n disulfide bond structures and may be a CH3 end of the repeating structural unit. The other end may also include n disulfide bond structures and may be a CH3 end from a structure other than the repeating structural unit. Here, the hydrophobic moiety may be attached to the one end, while the cancer cell recognition moiety may be attached to the other end.

In one embodiment of the present invention, a polymer compound containing a hydrophobic moiety that binds to natural killer cells, a cancer cell recognition moiety, and a linker containing the structure of chemical formula I was manufactured (Example 1, Example 2). The manufactured polymer compound was coated on the surface of natural killer cells (Example 1, Example 2), thereby enhancing the cancer cell recognition ability of the coated natural killer cells (Experimental Example 2), improving the cancer cell killing ability of the coated natural killer cells (Experimental Example 3 to Experimental Example 6), and confirming the cancer cell killing effect of the coated natural killer cells (Experimental Example 10, Experimental Example 11).

In one example of the manufactured polymer compound, natural killer cells were cultured in the presence of a component containing DSPE-PEDS_{5CFL-Arg-PEG1k}-FA conjugate to prepare natural killer cells with the DSPE-PEDS_{5CFL-Arg-PEG1k}-FA conjugate bound to their surface.

Analysis of the cancer cell recognition ability of natural killer cells coated with the DSPE-PEDS_{5CFL-Arg-PEG1k}-FA conjugate, according to the method of Experimental example 2, revealed that the cancer cell recognition ability of natural killer cells coated with the DSPE-PEDS_{5CFL-Arg-PEG1k}-FA conjugate from Example 1 was significantly enhanced by approximately 1.4 times compared to uncoated natural cells.

Analysis of the cancer cell cytotoxicity effect of natural killer cells coated with the DSPE-PEDS_{5CFL-Arg-PEG1k}-FA conjugate, according to the method of Experimental example 3, revealed a significant increase in the cytotoxicity effect of cancer cells when cultured with natural killer cells coated with the DSPE-PEDS_{5CFL-Arg-PEG1k}-FA conjugate from Example 1 compared to culturing uncoated natural killer cells with cancer cells. Furthermore, it was observed that as the number of natural killer cells coated with the DSPE-PEDS_{5CFL-Arg-PEG1k}-FA conjugate, increased, the enhancement of the cytotoxic effect on cancer cells upon co-culture also dramatically increased, indicating a progressive improvement in the cytotoxic effect with increasing coated natural killer cell numbers.

Analysis of the cancer cell cytotoxicity effect of natural killer cells coated with the DSPE-PEDS_{5CFL-Arg-PEG1k}-FA conjugate, according to the method of Experimental example 5, revealed that natural killer cells coated with the DSPE-PEDS_{5CFL-Arg-PEG1k}-FA conjugate induced more significant cancer cell death, accompanied by increased glutathione release.

Analysis of the cancer cell cytotoxicity effect of natural killer cells coated with the DSPE-PEDS_{5CFL-Arg-PEG1k}-FA conjugate, according to the method of Experimental example 6, revealed that the disulfide bonds of the DSPE-PEDS_{5CFL-Arg-PEG1k}-FA conjugate of Example 1 were cleaved, resulting in the release of folic acid (FA). After cleavage, the folate moiety, which serves as the cancer cell recognition moiety, was converted to an antifolate, further inducing the demise of cancer cells.

Analysis of the in vivo cancer cell cytotoxicity effect of natural killer cells coated with the DSPE-PEDS_{5CFL-Arg-PEG1k}-FA conjugate, according to the method of Experimental example 10, revealed that over time, the tumor volume in the experimental group injected with coated natural killer cells was relatively smaller compared to other control or experimental groups. The tumor growth inhibition rate was relatively higher, and the survival probability was also higher.

The analysis conducted according to the method of Experimental example 11, which involved histological examination to assess the enhanced anticancer efficacy of the cells, revealed that natural killer cells coated with the DSPE-PEDS_{5CFL-Arg-PEG1k}-FA conjugate exhibited an enhanced ability to infiltrate cancer cells, thereby demonstrating superior cytotoxic effects against cancer cells.

The DSPE-PEDS_{5CFL-Arg-PEG1k}-Biotin conjugate, an example of the polymer compound prepared, was used to culture natural killer cells under the component containing DSPE-PEDS_{5CFL-Arg-PEG1k}-Biotin. This resulted in the preparation of natural killer cells with the DSPE-PEDS_{5CFL-Arg-PEG1k}-Biotin conjugate bound to their surface.

The analysis of the anticancer effect on tumor cells was conducted on natural killer cells coated with the DSPE-PEDS_{5CFL-Arg-PEG1k}-Biotin conjugate according to the method of Experimental example 4. Results revealed a significant increase in the anticancer effect of the tumor cell line when cultured together with natural killer cells coated with the DSPE-PEDS_{5CFL-Arg-PEG1k}-Biotin conjugate, compared to the case where uncultured natural killer cells were cultured with the tumor cell line. Moreover, it was observed that as the number of natural killer cells coated with the PEDS_{5CFL-Arg-PEG1k}-Biotin conjugate, prepared according to Example 2 of the present invention, increased, the enhancement of the anticancer effect on the tumor cell line upon co-cultivation also dramatically increased.

The analysis of the anticancer effect on tumor cells was conducted on natural killer cells coated with the DSPE-PEDS_{5CFL-Arg-PEG1k}-Biotin conjugate according to the method of Experimental example 5. Results showed that natural killer cells coated with the DSPE-PEDS_{5CFL-Arg-PEG1k}-Biotin conjugate induced more tumor cell death and increased glutathione release.

In the present invention, “hydrophobic” refers to the property of having low affinity for water, which can enable high affinity for lipid molecules in biological systems. “Moieties” can refer to functional parts within a molecule.

In one embodiment of the present invention, a hydrophobic moiety within the polymer compound refers to a portion that exhibits hydrophobicity similar to the aforementioned property, while specifically binding to biomolecules. This part can have high affinity for lipids in biological systems, enabling it to be firmly attached (anchored) to the surface of natural killer cells.

In the present invention, the hydrophobic moiety is characterized by its hydrophobic properties, allowing it to bind or attach to the cell membrane. The hydrophobic moiety may be composed of low molecular weight substances, such as lipids, antibodies, hormones, drugs, and other molecules that can be administered in vivo, although it is not limited to these substances.

Specifically, in the present invention, the hydrophobic moiety can bind or attach to the lipid bilayer of immune cells, more specifically to the lipid bilayer of natural killer cells.

In one embodiment of the present invention, the polymer compound that recognizes natural killer cells and cancer cells may include a hydrophobic moiety that binds to natural killer cells. The hydrophobic moiety binding to natural killer cells may be any one of glycolipids with alkyl chains containing 12 to 24 carbon atoms, sterol lipids with 10 to 30 carbon atoms, 1,2-bis(diphenylphosphino)ethane (DPPE), or 1,2-bis (dimethylphosphino)ethane (DMPE). The lipid molecules of the cell membrane of the target natural killer cells for surface coating may have a structure similar to the lipid bilayer of the cell membrane.

The preferred glycolipids with alkyl chains containing 12 to 24 carbon atoms may include, but are not limited to, 1,2-distearoyl-sn-glycero-3-phosphoethanolamine (DSPE), dioleoylphosphatidylethanolamine (DOPE), distearoylphosphatidylcholine (DSPC), 1,2-dioleoyl-sn-glycero-3-phosphocholine (DOPC), dipalmitoylphosphatidylcholine (DPPC), dimyristoyl phosphatidylcholine (DMPC), dioleoyl-3-trimethylammonium propane (DOTAP), 1,2-distearoyl-3-trimethylammonium-propane chloride (DSTAP), and the like. However, it should be understood that this list is not exhaustive, and any lipid form of biomolecules that can be attached to and immobilized on the surface of natural killer cells and dissolved in nonpolar solvents can be used without limitation.

The preferred sterol lipids with alkyl chains containing 10 to 30 carbon atoms may include, but are not limited to, cholesterol, cholesterol hexacosanoate, 3ß-dimethylaminoethanol carbamoyl cholesterol, ergosterol, stigmasterol, or lanosterol. However, it should be understood that this list is not exhaustive, and any lipid form of biomolecules that can be attached to and immobilized on the surface of natural killer cells and dissolved in nonpolar solvents can be used without limitation.

As observed in Embodiment 1 of the present invention, the DSPE-PEDS5CFL-Arg-PEG1k-Biotin conjugate partially incorporates 1,2-Distearoyl-sn-glycero-3-phosphoethanolamine (DSPE), where DSPE is a compound with a hydrophobic chain attached in the form of consecutive CH2 units, suitable for anchoring to the surface of natural killer cells.

The hydrophobic moiety constituting the polymer compound for surface modification of natural killer cells in the present invention attaches to the surface of natural killer cells, while the cancer cell recognition moiety enhances the ability of coated natural killer cells to recognize receptors on the surface of cancer cells, thereby enhancing the breakdown or elimination capability of cancer cells.

The enhanced cancer cell recognition ability as described above is attributed to a mechanism where the structure of the cancer cell membrane is disrupted by the enzymatic action of natural killer cells, causing glutathione (GSH) inside the cancer cell to leak out into the extracellular space and cleave the disulfide bonds of the polymer compound. This process can be termed as a responsive unmasking process based on the reductive conditions in the tumor microenvironment.

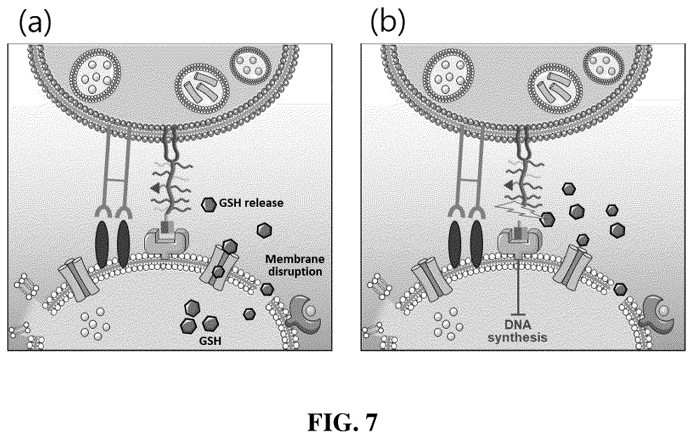

In one embodiment of the present invention, it is observed that the cleavage of disulfide bonds of the polymer compound leads to the attachment of the cancer cell recognition moiety at the end of the polymer compound. This moiety can bind to receptors on the surface of cancer cells, inducing the apoptosis of cancer cells (FIG. 7).

In other words, the polymer compound of the present invention, which recognizes natural killer cells and cancer cells, coats the cell surface by binding to the lipid bilayer of natural killer cells through hydrophobic moieties. Additionally, it can enhance the ability of natural killer cells to induce apoptosis of cancer cells by specifically recognizing cancer cells through cancer cell recognition moieties.

Furthermore, leakage of glutathione present within cancer cells due to cancer cell apoptosis can disrupt the disulfide bonds of the polymer compound of the present invention, converting the cancer cell recognition moiety into an antifolate form, thereby inhibiting DNA replication in cancer cells and inducing further cancer cell apoptosis.

The surface-modified natural killer cells with the polymer compound of the present invention exhibit enhanced cancer cell recognition capability, leading to a significant increase in cancer cell apoptosis through the release of lytic granules and cytokines. Moreover, the release of glutathione from within cancer cells further enhances cancer cell apoptosis by cleaving the cancer cell recognition moiety, contributing to its improvement.

The polymer compound of the present invention, which recognizes natural killer cells and cancer cells, may include a cancer cell recognition moiety. The cancer cell recognition moiety may comprise one or more selected from the group consisting of folic acid, biotin, phenylboronic acid, and phenylboronic acid, and preferably may be folic acid or biotin.

The polymer compound of the present invention, which recognizes natural killer cells and cancer cells, may include a linker comprising the structure of Chemical formula 1, to which one or more compounds selected from cell internalization inhibitors, cationic amino acids, and fluorescent dye compounds may be bound.

The cell internalization inhibitor compound mentioned above may include one or more selected from a group consisting of polyethylene glycol (PEG), polyethylene oxide (PEO), polyvinyl alcohol (PVA), and their copolymers.

The cell internalization inhibitor compound mentioned above can function to prevent the internalization of the polymer compound recognizing natural killer cells and cancer cells, maintaining attachment to the cell membrane surface.

In the polymer compound recognizing natural killer cells and cancer cells of the present invention, the cationic amino acid bound to the linker containing the structure of Chemical formula 1 may be selected from the group consisting of arginine, lysine, and histidine.

The aforementioned cationic amino acids can facilitate the approach to natural killer cells through electrostatic interactions.

The fluorescent dye compound bound to the linker containing the structure of Chemical formula 1 in the polymer compound recognizing natural killer cells or cancer cells, when recognized, enables the diagnosis or detection of target cells or cancer through fluorescence imaging.

The fluorescent dye compound may include fluorescent dyes based on the basic skeleton of rhodamine, coumarin, EvoBlue, oxazine, carbopyronine, naphthalene, biphenyl, anthracene, phenanthrene, pyrene, or carbazole, or derivatives thereof, exhibiting fluorescent properties and the fluorescent dye compounds exhibiting fluorescent properties may be used without limitation.

The polymer compound described herein may be characterized by its ability to bind to the surface of natural killer cells without impairing their viability and proliferation. Specifically, coating natural killer cells with the polymer compound prepared in one embodiment of the present invention did not affect the survival of natural killer cells. Furthermore, it was confirmed that coating the surface of natural killer cells with the polymer compound prepared in one embodiment of the present invention at concentrations up to 2 mg/mL did not impact the viability of natural killer cells. Moreover, it was observed that natural killer cells coated with the polymer compound at this concentration continued to proliferate well for up to 48 hours (FIG. 5).

Moreover, even when the polymer compound binds to the surface of natural killer cells, the native signaling receptors present on the cell membrane of natural killer cells can maintain their intrinsic characteristics, allowing them to interact appropriately with corresponding ligands.

This implies that attaching the polymer compound to the surface of natural killer cells does not exert any physical or biological effects on the activity of various receptors naturally present on the surface of natural killer cells, thereby allowing the normal functioning of cellular signaling mechanisms through these receptors.

Lipopolysaccharide (LPS) is known to bind to Toll-like receptors (TLRs) on the surface of natural killer cells, triggering intracellular signaling mechanisms that lead to the release of interferon-gamma (IFN-γ).

In one embodiment of the present invention, it has been confirmed that even when the polymer compound is attached to the surface of natural killer cells, the release of interferon-gamma (IFN-γ) occurs normally, indicating that the signaling mechanism mentioned above functions properly. This can be observed through the experimental results presented in FIG. 6.

The method of producing the polymer compound that recognizes natural killer cells and cancer cells according to the present invention comprises: (a) providing a compound represented by Chemical formula 3 by coupling a compound represented by Chemical formula 2, which is a hydrophobic moiety at one end, to a compound represented by Chemical formula 1; (b) coupling a cancer cell recognition moiety to the other end of the compound represented by Chemical Formula 3. However, it should be noted that the invention is not limited to these steps.

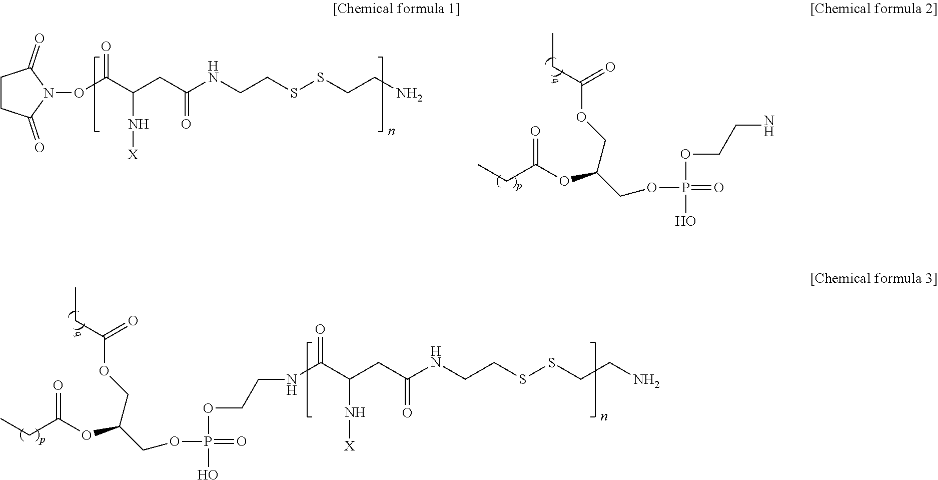

Here, the variable n is an integer ranging from 30 to 50, and the variable X represents one or more selected from acetyl, trifluoroacetyl, t-butoxycarbonyl (BOC), benzylcarbonyl (CBZ), and 9-fluorenylmethoxycarbonyl (Fmoc). Additionally, p is a natural number ranging from 12 to 20, and q is a natural number ranging from 12 to 20.

If “p” or “q” is less than 12, there may be a problem where the length of the lipid attached to the polymer compound is shortened, leading to a decrease in the attachment capability to natural killer cells. Conversely, if “p” or “q” exceeds 20, it may lead to steric hindrance or the formation of micelles before coating, which can impair the coating efficiency.

In an embodiment of the present invention, the method for manufacturing a polymer compound that recognizes natural killer cells and cancer cells may further include a step of substituting X in Chemical formula 1 with a compound for preventing cellular internalization, an amino acid with a positive charge, or a fluorescent dye compound.

In an embodiment of the present invention, the method for manufacturing a polymer compound that recognizes natural killer cells and cancer cells, the cancer cell-targeting moiety bound to the other end of Chemical formula 3 may be selected from the group consisting of folic acid, biotin, phenylboronic acid, and phenylboronic acid.

In an embodiment of the present invention, in the method for manufacturing a polymer compound that recognizes natural killer cells and cancer cells, the cell internalization prevention compound that can be coupled to X in Chemical formula 1 may be selected from the group consisting of polyethylene glycol (PEG), polyethylene oxide (PEO), polyvinyl alcohol (PVA), and their copolymers.

In an embodiment of the present invention, in the method for manufacturing a polymer compound that recognizes natural killer cells and cancer cells, the cationic amino acid that can be coupled to X in Chemical formula 1 may be selected from the group consisting of arginine, lysine, and histidine.

In an embodiment of the present invention, a pharmaceutical composition for cancer prevention or treatment comprises a hydrophobic moiety that binds to natural killer cells; a cancer cell recognition moiety; and a linker containing the structure of Chemical formula 1. At one end of Chemical formula 1, the hydrophobic moiety is bound, and at the other end, the cancer cell recognition moiety is bound, thereby including a polymer compound that recognizes natural killer cells and cancer cells as an active ingredient.

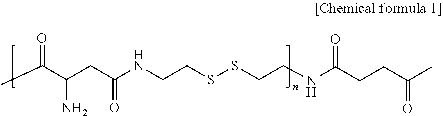

Here, the variable “n” is an integer ranging from 30 to 50.

Here, the pharmaceutical composition for cancer prevention or treatment, including the polymer compound recognizing natural killer cells and cancer cells as an active ingredient, can prevent or treat one or more cancer diseases selected from the group consisting of prostate cancer, thyroid cancer, stomach cancer, colon cancer, lung cancer, breast cancer, liver cancer, pancreatic cancer, testicular cancer, oral cancer, basal cell carcinoma, brain tumors, gallbladder cancer, bile duct cancer, laryngeal cancer, retinoblastoma, sarcoma, bladder cancer, peritoneal cancer, adrenal cancer, non-small cell lung cancer, esophageal cancer, small cell lung cancer, colon cancer, meningioma, esophageal cancer, urothelial cancer, kidney cancer, malignant bone tumors, malignant connective tissue tumors, malignant lymphomas, malignant melanoma, ocular tumors, urethral cancer, gastric cancer, sebaceous gland tumors, squamous cell carcinoma, hematological cancer, and anal cancer.

In an embodiment of the present invention, the pharmaceutical composition for cancer prevention or treatment, comprising the polymer compound recognizing natural killer cells and cancer cells as an active ingredient, may additionally incorporate a pharmaceutically acceptable carrier, excipient, or diluent.

In the context of the invention, the term “pharmaceutically acceptable” denotes characteristics indicating non-toxicity to cells exposed to the composition or to humans. Compositions containing pharmaceutically acceptable carriers may encompass various oral or non-oral formulations. In formulation, they may be prepared using diluents or excipients commonly employed, such as fillers, binders, lubricants, disintegrants, surfactants, or excipients. Examples of carriers, excipients, and diluents include but are not limited to lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, physiological saline, methylparaben, propylparaben, talc, magnesium stearate, mineral oil, dextrin, calcium carbonate, propylene glycol, and liquid paraffin. The aforementioned components may be used alone or in combination with each other, and are not limited to those listed. They can be incorporated independently or in combination with the active ingredient, the polymer compound, in the usual carriers, excipients, or diluents.

The pharmaceutical composition mentioned above may take any of the forms selected from the group consisting of solutions for injection, infusions, tablets, capsules, powders, granules, troches, suspensions, emulsions, syrups, sterile solutions for injection, non-aqueous solvents, suspensions, emulsions, suppositories, freeze-dried preparations, and lozenges.

The suppository base may include substances such as Witepsol, Macrogol, Tween 60, cocoa butter, laurin, glycerol gelatin, and the like.

The pharmaceutical composition may have any one of the forms selected from suppositories, granules, tablets, capsules, and liquid forms.

Additionally, The invention further provides a method for treating cancer, comprising administering a pharmaceutical composition comprising a polymer compound characterized by binding to natural killer cells, comprising a lipophilic moiety; a cancer cell recognition moiety; and a linker comprising the structure of Chemical formula 1, wherein said lipophilic moiety is bound to one end of said linker, and said cancer cell recognition moiety is bound to the other end of said linker, thereby recognizing natural killer cells and cancer cells.

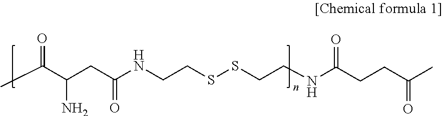

Herein, the value of “n” is an integer ranging from 30 to 50.

Here, the pharmaceutical composition comprising the polymer compound can be administered orally, either in solid or liquid form. Solid formulations for oral administration may include tablets, powders, granules, or capsules, prepared with at least one excipient such as starch, calcium carbonate, sucrose, lactose, or gelatin. Lubricants like magnesium stearate and talc may also be used besides simple excipients. Liquid formulations for oral administration encompass suspensions, solutions, emulsions, or syrups, often containing water or liquid paraffin as diluents, along with various excipients such as wetting agents, sweetening agents, flavoring agents, and preservatives. Formulations for non-oral administration may comprise sterile solutions, non-aqueous vehicles, suspensions, emulsions, freeze-dried formulations, or suppositories. Non-aqueous vehicles and suspending agents may include propylene glycol, polyethylene glycol, vegetable oils such as olive oil, injectable esters like ethyl oleate, and others.

The pharmaceutical composition of the present invention can be administered in pharmacologically effective amounts. There are no specific constraints on the dosage, as it may vary depending on factors such as absorption in the body, weight, age, gender, health status, diet, time of administration, method of administration, rate of excretion, and severity of the condition. The pharmaceutical composition of the present invention should be formulated considering the range of effective doses. The unit dosage forms thus formulated may be administered as needed, monitored, or observed by healthcare professionals based on individualized dosing regimens tailored to meet the requirements of the patient. Administration may occur once daily or divided into multiple doses as deemed appropriate, ensuring optimal therapeutic outcomes.

The term “said subject” refers to a target in need of disease treatment, more specifically encompassing mammals (for example, dogs, cats, horses, rabbits, zoo animals, cattle, pigs, sheep, and other non-human animals, as well as non-human primates). In specific embodiments, said subject in the present disclosure refers to a human.

### INDUSTRIAL APPLICABILITY

The present invention relates to a polymer compound capable of enhancing the anticancer function of natural killer cells by attaching to the surface of said cells, and a method for producing the same. By incorporating said polymer compound into pharmaceutical compositions, significant improvements in the prevention or treatment of cancer diseases can be achieved, thereby indicating industrial applicability.

1. A polymer compound characterized by comprising: a hydrophobic moiety binding to natural killer cells; a cancer cell recognition moiety; and a linker comprising the structure of Chemical formula 1, wherein the hydrophobic moiety is bound to one end of the linker, and wherein the cancer cell recognition moiety is bound to the other end of the linker, thereby forming a polymer compound capable of recognizing natural killer cells and cancer cells, 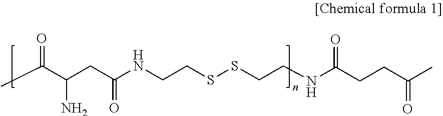
wherein, the variable “n” is an integer ranging from 30 to 50.

2. The polymer compound recognizing natural killer cells and cancer cells according to claim 1, wherein the hydrophobic moiety binds to natural killer cells, and said cancer cell recognition moiety recognizes cancer cells, thereby promoting apoptosis of cancer cells.

3. The polymer compound recognizing natural killer cells and cancer cells according to claim 1, wherein the hydrophobic moiety binding to the natural killer cells comprises a lipid with an alkyl chain containing 12 to 24 carbon atoms; a sterol lipid with 10 to 30 carbon atoms; 1,2-bis(diphenylphosphino)ethane (DPPE); or 1,2-bis (dimethylphosphino)ethane (DMPE).

4. The polymer compound recognizing natural killer cells and cancer cells according to claim 1, wherein the cancer cell-targeting moiety comprises one or more selected from the group consisting of folic acid, biotin, phenylboronic acid, and phenylboronic acid.

5. The polymer compound recognizing natural killer cells and cancer cells according to claim 1, wherein a polymer compound recognizing natural killer cells and cancer cells, comprising a linker incorporating a compound for intracellular trafficking prevention, one or more cationic amino acids, and a fluorescent dye compound, all of which are associated with the structure of chemical formula 1.

6. The polymer compound recognizing natural killer cells and cancer cells according to claim 5, wherein the compound for intracellular trafficking prevention, as described above, is selected from the group consisting of polyethylene glycol (PEG), polyethylene oxide (PEO), polyvinyl alcohol (PVA), and their copolymers.

7. The polymer compound recognizing natural killer cells and cancer cells according to claim 5, wherein the cationic amino acid is selected from the group consisting of arginine, lysine, and histidine.

8. A preparation method of a polymer compound recognizing natural killer cells and cancer cells, the method comprising: (a) providing a compound represented by the following chemical formula 3 by coupling a compound represented by the chemical formula 2 to one end of a compound represented by the following chemical formula 1; and (b) a step of coupling an cancer cell recognition moiety to the other end of a compound represented by the chemical formula 3, 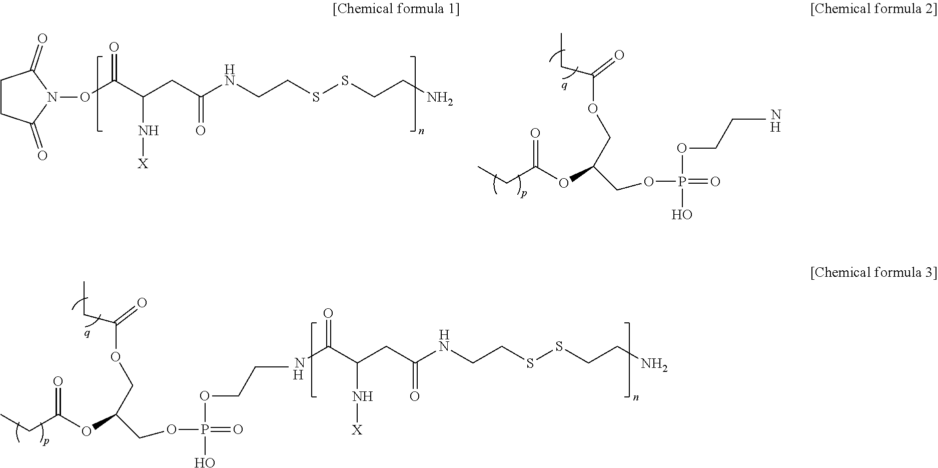
wherein, the variable n is an integer ranging from 30 to 50, and the variable X represents one or more selected from acetyl, trifluoroacetyl, t-butoxycarbonyl (BOC), benzylcarbonyl (CBZ), and 9-fluorenylmethoxycarbonyl (Fmoc). Additionally, p is a natural number ranging from 12 to 20, and q is a natural number ranging from 12 to 20.

9. A preparation method of a polymer compound recognizing natural killer cells and cancer cells according to claim 8, wherein the method for producing a polymer compound that recognizes natural killer cells and cancer cells, further comprising the step of substituting X with one of a compound for preventing cellular internalization, cationic amino acids, or fluorescent dye compounds.

10. A preparation method of a polymer compound recognizing natural killer cells and cancer cells according to claim 8, wherein the cancer cell recognition moiety is selected from a group consisting of folic acid, biotin, phenylboronic acid, and phenylboronic acid.

11. A preparation method of a polymer compound recognizing natural killer cells and cancer cells according to claim 8, wherein the cell internalization prevention compound is selected from a group consisting of polyethylene glycol (PEG), polyethylene oxide (PEO), and their copolymers.

12. A preparation method of a polymer compound recognizing natural killer cells and cancer cells according to claim 8, wherein the cationic amino acid is selected from a group consisting of arginine, lysine, and histidine.

13. The polymer compound recognizing natural killer cells and cancer cells, manufactured through the method according to claim 8.

14. The polymer compound recognizing natural killer cells and cancer cells, manufactured through the method according to claim 9.

15. The pharmaceutical composition for preventing or treating cancer including a polymer compound characterized by comprising: a hydrophobic moiety binding to natural killer cells; a cancer cell recognition moiety; and a linker comprising the structure of Chemical formula 1, wherein the hydrophobic moiety is bound to one end of the linker, and wherein the cancer cell recognition moiety is bound to the other end of the linker, thereby forming a polymer compound capable of recognizing natural killer cells and cancer cells, 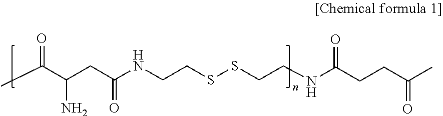
wherein the variable “n” is an integer ranging from 30 to 50.

16. The pharmaceutical composition for preventing or treating cancer according to claim 15, wherein the diseases for which the anticancer effect is achieved by the pharmaceutical composition include, but are not limited to, prostate cancer, thyroid cancer, stomach cancer, colorectal cancer, lung cancer, breast cancer, liver cancer, pancreatic cancer, testicular cancer, oral cancer, basal cell carcinoma, brain tumor, gallbladder cancer, bile duct cancer, laryngeal cancer, retinoblastoma, Burkitt's lymphoma, bladder cancer, peritoneal cancer, adrenal cancer, non-small cell lung cancer, esophageal cancer, renal pelvis and ureter cancer, kidney cancer, malignant bone tumors, malignant soft tissue tumors, malignant lymphoma, malignant melanoma, eye tumor, urethral cancer, gastric cancer, melanoma, cervical cancer, endometrial cancer, uterine fibroids, metastatic brain tumors, colorectal cancer, vaginal cancer, spinal tumor, salivary gland cancer, tonsil cancer, squamous cell carcinoma, blood cancer, and anal cancer.

17. The pharmaceutical composition for preventing or treating cancer according to claim 15, wherein the composition further comprises pharmaceutically acceptable carriers, excipients, or diluents.

18. The method for treating cancer according to claim 15, where in the comprising administering to a subject a pharmaceutical composition.

19. The method for treating cancer according to claim 16, where in the comprising administering to a subject a pharmaceutical composition.

20. The method for treating cancer according to claim 17, where in the comprising administering to a subject a pharmaceutical composition.
